(19) United States
(12) Patent Application Publication
(10) Pub. No.: US 2018/0255771 A1
(43) Pub. Date: Sep. 13, 2018

(54) CONTROLLED GAS PHASE RELEASE OF NON AQUEOUS PHASE LIQUID ESSENTIAL OIL COMPOUNDS FOR AROMATHERAPY, FRAGRANCE AND INSECTICIDAL ACTIVITY

(71) Applicant: George Edward Hoag, Storrs, CT (US)

(72) Inventor: George Edward Hoag, Storrs, CT (US)

(21) Appl. No.: 15/764,923

(22) PCT Filed: Sep. 29, 2016
(86) PCT No.: PCT/US2016/054476

§ 371 Date: Mar. 30, 2018

Related U.S. Application Data
(60) Provisional application No. 62/234,975, filed on Sep. 30, 2015.

(51) Int. Cl. A01N 25/18; A01N 25/34; A01N 25/10; A61L 9/04; A01N 65/00; A01N 65/22; A01N 65/28; A01N 65/08; A01N 65/24; A01N 65/44

(52) U.S. Cl. CPC A01N 25/18; A01N 25/34; A01N 25/10; A61L 9/042; A01N 65/44; A01N 65/22; A01N 65/28; A01N 65/08; A01N 65/24; A01N 65/00

(57) ABSTRACT

This disclosure relates to devices, methods and compositions for the controlled gas phase time-release of volatile and semi-volatile hydrophobic organic compounds from a granular polymer sorbent-non aqueous phase liquid-porous media matrix. Also, this disclosure relates to devices, methods and compositions for steady and long-term release into the gas phase of volatile organic compounds from a granular polymer sorbent-essential oil matrix coating made at ambient temperatures and pressures and placed in the porosity of granular porous media materials such as plastic particles, plastic absorption or stripping tower mass transfer packing material, wood pellets and geologic material. The gas phase compounds released from the granular polymer sorbent-essential oil matrix have aromatherapy, fragrance, insecticidal and insect repelling properties.

### RELATED APPLICATIONS

This application is a national phase application claiming the benefit of PCT International Application No. PCT/US2016/054476, filed on Sep. 29, 2016, which is incorporated herein by reference.

### BACKGROUND OF THE DISCLOSURE

### 1. Field of the Disclosure

This disclosure relates to devices, methods and compositions for the controlled gas phase time-release of volatile and semi-volatile hydrophobic organic compounds from a granular polymer sorbent-non aqueous phase liquid-porous media matrix. More particularly this disclosure relates to devices, methods and compositions for steady and long-term release into the gas phase of volatile organic compounds from a granular polymer sorbent-essential oil matrix coating made at ambient temperatures and pressures and placed in the porosity of granular porous media materials such as plastic particles, plastic absorption or stripping tower mass transfer packing material, wood pellets and geologic material. Even more particularly, the gas phase compounds released from the granular polymer sorbent-essential oil matrix have aromatherapy, fragrance, insecticidal and insect repelling properties.

### 2. Description of the Related Art

The controlled release of volatile and semi-volatile organic compounds into the gas phase is a key factor in aromatherapy, fragrance, insecticidal and insect repellent products. There are some applications for the controlled release into the gas phase of volatile and semi-volatile organic compounds where the rate of release of these compounds is desired to be rapid and others where the rate of release is desired to be slow, in the order of days, weeks, months or years. Fumigation of insects or other pests in order to kill or inactivate them is an application in which the rate of gas phase release is desired to be rapid to provide a bolus dose for effective treatment. In other cases it is desired to have a slow sustained release rate of volatile or semi-volatile organic compounds into the gas phase for the purpose of aromatherapy, increased odor longevity benefits of fragrances and perfumes, insect repellency and longer term insecticidal applications. One feature of this present disclosure are compositions and methods to provide controlled and sustained release of volatile and semi-volatile non aqueous phase liquid organic compounds into the gas phase from a granular polymer sorbent-essential oil matrix for a period from about 1 to about 100 days. Another feature of this disclosure are compositions and methods to provide controlled release, either passively or actively with an air blower or air pump to provide controlled sustained release of volatile and semi-volatile non aqueous phase liquid organic compounds into the gas phase from an absorption or stripping type mass transfer packing material granular polymer sorbent-essential oil matrix for a period from about 1 to about 100 days. The absorption or stripping type mass transfer packing materials are commonly and commercially available, for example but not limited to Flexiring™, Intalox™ Snowflake, Cascade Mini-Rings™, BetaRings™ manufactured by Koch-Glitsch LP, Jaeger tri-Paks® manufactured by Rashig USA, Inc., Coralife® Bio-Balls, or Marineland Bio-Filter Balls. Non-aqueous phase liquid organic compounds are defined in this patent as organic compounds that are immiscible in water, have limited solubility in water, are hydrophobic and have a vapor pressure greater than 0.0002 mm Hg at 20° C.

There have been many developments for both active and passive release of organic compounds into the gas phase from Non Aqueous Phase Liquids. U.S. Pat. No. 8,968,647 (Fischer et al.) describes a passive multi-layer article fragrance delivery system that is claimed to provide a constant olfactive effect for up to two months or longer containing a one or more sorbent layers, one or more vapor permeable or microporous layers and optionally, a vapor impermeable layer adjacent to a portion of the sorbent layer not adjacent to the vapor permeable or microporous layer. The sorbent layer contains 60% by weight of at least one volatile component having a vapor pressure of 0.03 to 40.0 mm Hg when measured at 20° C. and 760 mm Hg; wherein the sorbent layer is a nonwoven material having a density from 2.0 to 25 mg/cm³. Each layer is capable of being formed into a required shape or structure. The disclosure includes compositions and methods for its use in treating the atmosphere of an environment such as a compartment or localized space.

WO 93/00115 (Fischel-Ghodsian) (Jan. 7, 1993) describes a device for controlled release of vaporous medications or repellants that is a multilayered laminate having, in order from the rear, an adhesive layer for fixing the device to a surface, an impermeable membrane layer, a reservoir layer incorporating the active material and a diffusion rate limiting layer on the front surface. The impermeable layer is described as a metal foil polymer laminate, wherein the polymer is selected from polyethylene terephthalate, polyethylene, polypropylene, polyvinyl chloride and ethylene vinyl acetate. The reservoir layer is made of a porous polymer, an impregnated polymer or a gelled polymer mixture.

U.S. Pat. No. 5,820,791 (Canale) describes an air freshening device used in conjunction with an air circulation system of a building for impregnating the air with an aromatic scent. The device comprises a combination integrally-mixed plastic and scent substance formed through a manufacturing process that produces a shape for the scent impregnating device which permits it to be removeably attached to an air filter.

U.S. Pat. No. 4,604,114 (Ward) discloses a fragrant scented air filter air freshening device particularly adapted for use in conjunction with interior building air conditioning systems. In this system scented extruded plastic beads containing an air freshening material in an air permeable envelope and a fastener attached at one end to the air permeable envelope adapted for securing the envelope to an air filter air freshening device to the air filter.

WO 03/097114 (Jean et al.) describes a molded thermoplastic article consisting of a decorative face and a plastic replaceable element that is impregnated with a volatile active ingredient such as a perfume. The resin of the element is a volatile impregnated polyether-ester-amide type plastic that is permeated with a volatile active ingredient. The element is attached to a fixing system, which associates the device with a ventilation or air conditioning system.

U.S. Pat. No. 7,159,792 (Wheatley et al.) describes an air freshener that can be removeably adhesively attached to a surface. The device consists of a scent portion and attachment portion. The scent portion contains an active carrier material, such as a polymer or polymer gel that is freestanding and contains an active, such as scented oil. The polymer body is compressible under an applied force and substantially returns to its original shape when the force is removed. Polyurethane or polyurethane gel is described as an example of a carrier material. The attachment portion includes a pad formed of a material that imparts a “clingy, tacky or frictional quality,” which provides surface adhesion. The shape and size of the scent portion can be configured to determine the amount and rate of scent release.

U.S. Pat. No. 8,931,711 (Gruenbacher, et al.) describes an apparatus for passive delivering a volatile material in a continuous manner. The apparatus includes a delivery engine having a reservoir for containing a volatile material; a rupturable substrate secured to the reservoir; a rupture element positioned adjacent to the rupturable substrate; and a breathable microporous membrane wherein said comprising an average pore size of about 0.01 microns to about 0.06 microns said delivery engine provides 30 to 90 days of continuous emission of said volatile material without leakage.

U.S. Pat. No. 8,822,533 (Chauhan, et al.) describes unique volatile pyrethroids and a method for repelling and/or killing insects involving treating an object or area with an insect repelling effective amount or insect killing effective amount of at least one unique volatile pyrethroid and optionally a carrier, wherein the object or area is clothing or items attached to clothing and the carrier component can be a liquid or a solid material. The vehicle or carrier to be used refers to a substrate such as a gel or polymers.

U.S. Pat. No. 8,695,891 (Santini, et al.) describes a fragrance delivery device comprising a hollow vessel containing a fragrance medium and further comprising a porous exterior surface with a porous wicking extension and a reservoir containing a second fragrance medium with the second fragrance medium in fluid communication with the wicking extension and the porous exterior surface. The first fragrance medium is selected from a candle, a gel composition, fragranced membranes, polymer beads, a liquid fragrance medium with an exposed wick, and fragranced potpourri. It is further described that fragranced air freshening gels can be formed by the use of either naturally derived hydrocolloids such as carrageenan or gellan gums for use in aqueous based compositions, metallic salts such as sodium stearate for water, glycol compositions, or through the use of synthetic polymers such as thermoplastic elastomers, polyurethane and polyamides, for anhydrous or substantially anhydrous fragrance solvent systems. Alternatively, another fragrance medium could be housed in the hollow vessel, such as, but not limited to, potpourri, fragranced soaps, and fragranced polymer beads. The gels can be poured into the hollow vessel either as heated liquefied fluids such that upon cooling they set into a final solid or semi-solid form or they can be poured at ambient temperature and through a cross-linking process develop into their final gel-like consistency over a period of time.

U.S. Pat. No. 5,871,765 (Johnson, et al.) describe and article of manufacture comprising a non-aqueous, controlled-release air care composition comprising from about 20 to about 95 weight percent of a volatile hydrocarbon with one or more hydrocarbon-soluble substances and from about 1 to about 50 weight percent of a blend of diblock and triblock, radial block and/or multiblock copolymers and from about 0.1 to about 30 weight percent of one or more air care active substances, wherein said one or more active substances are released into the air by evaporation, and wherein said active substances are selected from the group consisting of nematocides and fungicides.

U.S. Pat. No. 7,470,738 (Sumiya, et al.) describes anon-aqueous absorbent comprising a specific crosslinked polymer containing a structural unit having a carboxyl group and/or a sulfonate group in an amount of 20 to 100% by weight in its molecule and in which from 30 to 100% by mol of the protons in the carboxyl group and/or the sulfonate group have been substituted by onium cations. A non-aqueous gel comprising the non aqueous sorbent and an organic solvent is also described in the disclosure. Further an insecticidal composition or an insecticide is described, which comprises the non-aqueous absorbent and a pyrethroid insecticidal ingredient.

U.S. Pat. No. 7,273,184 (Brown, et al.) describes an air treatment device, comprising a hanger to secure an air treatment device to a garbage bin with a volatile material dispenser adapted to dispense a volatile material to the interior of a garbage bin, where the volatile material dispenser is an openable container that comprises a cover shell and an absorbent material-containing body that is enclosed within the shell. A planar support element is a volatile material comprising a non-porous material defining an open cavity containing an absorbent material within the cavity and held therein by a porous retaining member that prevents the absorbent material from exiting the cavity but permits the passage of volatile material entrained or absorbed by the absorbent material through the porous retaining member.

U.S. Pat. No. 8,684,739 (Steffier, et al.) describes method for sustained delivery of an active ingredient into a film coating and sustained delivery homogeneously-dispersed, porous polymeric or co-polymeric beads, the beads having a continuous network of interconnecting pores, comprising macropores; mesopores, micropores of <20 Angstoms diameter, and gel porosity, and wherein the at least one active ingredient is contained within the network of pores; and applying the mixture in fluid or gel form to a surface, thereby forming a film coating, which continuously transports outward the at least one active ingredient by internal flow within the network of pores, resulting in delivery from the macropores to the coated surface for a sustained time of at least 24 hours from application, and wherein the network of pores is non-collapsible upon removal of the active ingredient.

U.S Patent Application No. 20140335140 (Hoag, et al.) describes a composition and method that to repelling insects, arachnids and other arthropods by the slow release volatilization of essential oils into a gas phase, comprising: one or more essential oils or their extracts; one or more natural waxes; a support material onto which a matrix of the essential oil and the natural wax is solidified; and a housing or container that retains the essential oil-wax matrix on the support material with sufficient air permeability or diffusivity to allow volatilization, mass transfer and diffusion of the volatile fraction of the essential oil from the essential oil-wax matrix.

Increasingly natural or naturally derived organic compounds have been identified that repel insect as well, and in some cases, better than N,N-diethyl-meta-toluamide (DEET). For example, Del Fabbro, S. and Nazzi. F. (2013) from Chemistry to Behavior. Molecular Structure and Bioactivity of Repellents against Ixodes ricinus Ticks. Plos One, 8, 6, e67832, reported that 6 compounds related to eugenol, as well as eugenol at a 100 ug dose were as effective at repelling ticks (Ixodes ricinus) as DEET at a 100 ug dose. They also reported that 3 compounds (4-Allyl-2,6-dimethoxyphenol (2,6-Dimethoxy-4-allylphenol), 4-Methyl-1,2-benzenediol (4-Methylcatechol) and 4-Ethyl-1,2-benzenediol (4-Ethylcatechol)) with similar structures to eugenol were as repellent as DEET at a 10 ug dose. Indeed 4-Ethyl-1,2-benzenediol (4-Ethylcatechol) was reported to be statistically significantly more repellent to Ixodes ricinus at a 1 ug dose than DEET which was not reported to be effective at a 1 ug dose.

Synthetic insect repelling and insecticidal compounds are numerous and compounds with vapor pressures greater than 0.0002 mm Hg at 20° C. are volatile enough to be used in this present disclosure.

Pyrethrum is a natural insecticide made from the dried flower heads of Chrysanthemum cinerariifolium and Chrysanthemum coccineum. Pyrethrins consist of 6 compounds in pyrethrum that have insecticidal potency. PMD, (para-menthane-3,8-diol) is the major constituent of the byproduct from the distillation of leaves from the Australian lemon-scented gum tree, Corymbia citriodora. Both pyrethrins and PMD are very effective insect repellents to many insect species including ticks, mosquitoes and gnats. The United States Environmental Protection Agency (USEPA) Federal Insecticide, Fungicide and Rodenticide Act (FIFRA) under 40CFR 152.22 (g)(1) Appendix A USEPA has promulgated a list of minimum risk pesticide active and inactive components that can be insect repellent products that are exempt from registration [exempt under 40 CFR 152.25(f)]. USEPA lists several essential oils and essential oil extracts that are considered minimum risk pesticides, including; cedar oil, cinnamon oil, citronella oil, clove oil, eugenol, geraniol, geranium oil, lemongrass oil, mint oil, peppermint oil, rosemary oil and thyme oil. Additionally dozens of additional essential oils have been reported with insect repellency (Maia, M. F, and Moore, S. J. (2011) Plant-based insect repellents: a review of their efficacy, development and testing Malaria Journal, 10 (Suppl 1):S11, including; Corymbia citriodora, Eucalyptus spp., E. camaldulensis, E. globulous, Eugenia caryophyllus, Syzygium aromaticum, Eugenia aromaticum, Lippia spp, L. javanica, L. uckambensis, L. cheraliera, Lantana camara, Lavandula spp., L. angustifolia, L. latifolia, Melaleuca alternifolia, Ocimum spp. O. americanum, O. suave, O. basilicum, O. kilimandscharikum, O. forskolei, Hyptis spp. Hyptis suaveolens, Mentha spp. M. piperata, M. arvensis, Thymus spp. Th. vulgaris, Th. satureioides; Pogostemon spp, Pogostemon cablin, Cymbopogon spp., C. nardus, C. martini, C. citratus, C. winterianius, C. excavates, Pelargonium reniforme, Azadirachta indica, Tagetes minuta, Artemisia spp. A. vulgaris, A. monosperma, Daniellia oliveri, Glycine max, Zanthoxylum limonella, Citrus hystrix, Curcuma longa.

Common insect repelling compounds with vapor pressures greater than 0.0002 mm Hg at 20° C. include, but are not limited to DEET, picarin, methyl jasmonate, p-menthane-3,8-diol, menthofuran, thiophene, coumaran, butyl formate heptyl formate, hexyl formate, ethylene glycol diformate, dichlorvos and 2-undecanone.

The present disclosure provides many advantages, which shall become apparent as described below.

### SUMMARY OF THE DISCLOSURE

This disclosure relates in part to compositions and methods for the passive or active variable rate (slow to fast) sustained time-release of insect repelling, insecticidal, or fragrance emitting volatile organic compounds into the air. One embodiment of this present disclosure provides for a method and a device that is manufactured at ambient temperatures used to repels or inactivate insects or other arthropods by the passive, slow, sustained time-release of repelling, insecticidal or fragrance emitting volatile organic compounds into the air containing:
a) a volatile organic compound-granular polymer matrix consisting of one or more volatile organic compounds sorbed with one or more solid polymers;
b) naturally occurring or man-made porous or non-porous solid particles coated with the volatile organic compound-granular polymer matrix;
c) a gas permeable, gas semi-permeable or a microporous material on at least one side of the structure or container that contains the solid particles coated with the volatile organic compound granular-polymer-matrix; and optionally
d) a gas impermeable material on one or more sides of the material, structure or container that contains the solid particles coated with the volatile organic compound-granular polymer matrix.

This disclosure also relates in part to a method and a device that is manufactured at ambient temperatures used to repels or inactivate insects or other arthropods by the active and controllable rate of sustained time-release of repelling, insecticidal, or fragrance emitting volatile organic compounds into the air containing:
a) a volatile organic compound-granular polymer matrix consisting of one or more volatile organic compounds sorbed with one or more solid polymers;
b) man-made porous plastic, metal or ceramic mass transfer packing media coated or embedded with the volatile organic compound-granular polymer matrix and placed in a cylindrical, rectangular or cubical filter housing open on at least two ends;
c) an air blower or air pump with the air exhaust that passes through the filter housing and volatilizes the insect repelling, insecticidal or fragrance emitting volatile organic compounds into the air stream; and
d) a pipe or series of pipes or air handling ductwork that conveys and directs the insect repelling, insecticidal or fragrance emitting volatile organic compounds into enclosed, semi-enclosed or open air spaces.

This disclosure further relates in part to a method and a device that is manufactured at ambient temperatures used to provide an aromatherapy environment passive, slow, sustained time-release of aromatherapy or fragrance emitting volatile organic compounds into the air containing:
a) a volatile organic compound-granular polymer matrix consisting of one or more volatile organic compounds sorbed with one or more solid polymers;
b) naturally occurring or man-made porous or non-porous solid particles coated with the volatile organic compound-granular polymer matrix;
c) a gas permeable, gas semi-permeable or a microporous material on at least one side of the structure or container that contains the solid particles coated with the volatile organic compound granular-polymer-matrix; and optionally
d) a gas impermeable material on one or more sides of the material, structure or container that contains the solid particles coated with the volatile organic compound-granular polymer matrix.

This disclosure still further relates in part to a method and a device that is manufactured at ambient temperatures used to provide an aromatherapy environment by the active and controllable rate of sustained time-release of aromatherapy or fragrance emitting volatile organic compounds into the air containing:
a) a volatile organic compound-granular polymer matrix consisting of one or more volatile organic compounds sorbed with one or more solid polymers;
b) man-made porous plastic, metal or ceramic mass transfer packing media coated or embedded with the volatile organic compound-granular polymer matrix and placed in a cylindrical, rectangular or cubical filter housing open on at least two ends;
c) an air blower or air pump with the air exhaust that passes through the filter housing and volatilizes the insect repelling, insecticidal or fragrance emitting volatile organic compounds into the air stream; and
d) a pipe or series of pipes or air handling ductwork that conveys and directs the insect repelling, insecticidal or fragrance emitting volatile organic compounds into enclosed, semi-enclosed or open air spaces.

One embodiment of this present disclosure is an improvement from prior art in which aromatherapy, fragrances, perfumes, insecticides, essential oils or essential oil extracts have been made into gels with polymers or sorbed with polymers. In the prior art, the aromatherapy, fragrances, perfumes, insecticides, essential oils or essential oil extracts that have been made into gels or sorbed onto polymers are limited in surface area contact with a gas phase or ambient air and therefore limits the effectiveness of the mass transfer and diffusion of the volatile organic compounds associated with these materials into the gas phase or ambient air. As a result several applications in the prior art require a mechanical device, for example a blower or a heater to accelerate the mass flux of the volatile organic materials into the gas phase to achieve a desired effect. The mass flux of volatile organic compounds into a gas phase or the ambient air is controlled in part by the surface area exposed to a gas phase or the ambient air. In this embodiment of the present disclosure the large surface area associated with porous or non-porous particles coated with the aromatherapy, fragrance, perfume, insecticide, insect repelling essential oil extract-granular polymer matrix enables sufficient diffusion and mass transfer of volatile organic compounds associated with the aromatherapy, fragrance, perfume, insecticide, insect repelling essential oil extract-granular polymer matrix into a gas phase or ambient air passively, without the requirement of a mechanical device such as a heater or blower. Non-Aqueous Phase Liquid organic compounds are defined in this patent as organic compounds that are immiscible in water, have limited solubility in water, are hydrophobic and have a vapor pressure greater than 0.0002 mm Hg at 20° C.

In another embodiment of this present disclosure, coating or embedding of larger size structured or random porous 0.25 inch (6.35 mm) to 1.5 inch (38.1 mm) man-made porous plastic, metal or ceramic mass transfer packing media with the volatile organic compound-granular polymer matrix and placing the coated media in a cylindrical, rectangular or cubical filter housing and at least partially open on at least two ends provides for low air pressure drop across the filter and efficient mass transfer of the volatile organic compounds from the volatile organic compound-granular polymer matrix into the gas or air phase for effective delivery of the fragrance, perfume, insecticide, insect repelling volatile organic compounds into an enclosed, semi-enclosed or open space. The use of an air blower to air pump to create air flow rates from 1 cubic foot per minute to 10,000 cubic feet minute enables larger volume enclosed, semi-enclosed or open spaces to be effectively treated with aromatherapy, fragrance, perfume, insecticide, or insect repelling volatile organic compounds and at higher volatile organic compounds concentrations and at faster rates of emission than is possible with the passive release of aromatherapy fragrance, perfume, insecticide, insect repelling volatile organic compounds from the porous or non-porous particles coated solid particles placed in a passive release container.

In another embodiment of this present disclosure activated carbon, either granular or powdered, is incorporated into the aromatherapy, fragrance, perfume, insecticide, or insect repelling essential oil, fragrance oil, or essential oil extract-granular polymer matrix used by itself without coating porous or non-porous particles or by coating porous or non-porous solid particles.

In yet another embodiment of this present disclosure the aromatherapy, fragrance, perfume, insecticide, or insect repelling essential oil, fragrance oil, or essential oil extract-granular polymer matrix used by itself without coating porous or non-porous particles or by coating porous or non-porous solid particles can be placed in a structure that contains the aromatherapy or fragrance volatile organic compound-granular polymer matrix that can be opened to the atmosphere on one or more sides, as well as a structure that contains the aromatherapy or fragrance volatile organic compound-granular polymer matrix that consists of a gas impermeable material sealed or closed to the atmosphere on one or more sides.

Further objects, features and advantages of the present disclosure will be understood by reference to the following drawings and detailed description.

### BRIEF DESCRIPTION OF THE DRAWINGS

FIG. 1 is a table of ingredients and properties of the insect repellent device in accordance with Example 1.

FIG. 2 is a table of ingredients and properties of the insect repellent device in accordance with Example 2.

FIG. 3 is a table of ingredients of the aromatherapy essential oil matrix in accordance with Example 3.

FIG. 4 is a table of ingredients of the jasmine fragrance matrix in accordance with Example 4.

FIG. 5 is a table of ingredients of the insect repellent device composition in accordance with Example 5.

### DESCRIPTION OF THE EMBODIMENTS

In this present disclosure the mass flux of volatile organic compounds that volatilize from an aromatherapy, fragrance, perfume, insecticide, insect repelling essential oil, a fragrance oil or essential oil extract-granular polymer matrix into a gas phase or the ambient air is increased by the large surface area created by coating the aromatherapy, fragrance, perfume, insecticide, insect repelling essential oil, fragrance oil or essential oil extract-granular polymer matrix onto large surface area to volume ratio porous or non-porous solid particles. The large surface area to volume ratio porous or non-porous solid particles that are used as a support for the essential oil-granular polymer matrix have dimensions from about 1 mm to about 5 mm in either length, width or diameter and are either spherical, cylindrical, or cubical in shape, with variable surface roughness thereof and provides large surface area compared to monolithic shapes of used in the prior art. The use of larger 0.25 inch (6.35 mm) to 1.5 inch (38.1 mm) man-made engineered porous plastic, metal or ceramic mass transfer packing media coated or embedded with the volatile organic compound-granular polymer matrix enables even larger surface area to volume ratios than possible in comparison to other solid non-porous or porous materials.

The volatilization of the volatile organic compounds associated with the devices in this present disclosure from the aromatherapy, fragrance, perfume, insecticide, insect repelling essential oil, fragrance oil or essential oil extract-granular polymer matrix to a gas phase or the ambient air from the gas or air filled porosity between the aromatherapy, fragrance, perfume, insecticide, insect repelling essential oil, fragrance oil or essential oil extract-granular polymer matrix coated porous or non-porous solid particles is controlled by several composition variables in this present disclosure, containing:
a) the vapor pressure of one or more of the volatile organic compounds in the aromatherapy, fragrance, perfume, insecticide, insect repelling essential oil, fragrance oil or essential oil extracts;
b) the mole fractions, the vapor pressures and the weight ratios of the various organic compounds present in the non-aqueous phase liquid associate with the aromatherapy, fragrance, perfume, insecticide, insect repelling essential oil, fragrance oil or essential oil extracts, commonly described by Raoult's Law.
c) the weight ratio of the non-aqueous phase aromatherapy, fragrance, perfume, insecticide, insect repelling essential oil, fragrance oil or essential oil extract to polymer;
d) the type of polymer or mixture of polymers used to sorb or embed the non-aqueous phase aromatherapy, fragrance, perfume, insecticide, insect repelling essential oil, fragrance oil or essential oil extracts;
e) the weight and volume ratio of the aromatherapy, fragrance, perfume, insecticide, insect repelling essential oil, fragrance oil or essential oil extract-granular polymer matrix to the porous or non-porous solid particles;
f) the size of the porous or non-porous solid particles or man-made engineered porous plastic, metal or ceramic mass transfer packing media;
g) the surface area to volume ratio of the porous or non-porous solid particles or man-made engineered porous plastic, metal or ceramic mass transfer packing media;
h) the interparticle pore sizes;
i) the bulk density of the aromatherapy, fragrance, perfume, insecticide, insect repelling essential oil, fragrance oil or essential oil extract/granular polymer/solid or porous particle matrix;
j) the geometry, porosity and air permeability of the housing that contains aromatherapy, fragrance, perfume, insecticide, insect repelling essential oil, fragrance oil or essential oil extract/granular polymer/solid or porous particle matrix; and
k) the incorporation of the aromatherapy, fragrance, perfume, insecticide, insect repelling essential oil, fragrance oil or essential oil extract/granular polymer/porous particle matrix into an enclosed packed bed air filter allowing air from an air blower or an air pumped to advectively pass through the packed bed air filter enabling volatile organic carbon compounds to volatilize into the gas phase at a rate greater than possible with a passive system alone.

The larger surface area associated with the aromatherapy, fragrance, perfume, insecticide, insect repelling essential oil, fragrance oil or essential oil extract-granular polymer matrix coated or embedded particles in this present disclosure results in a composition with a greater and more controllable diffusional and mass transfer flux of volatile organic compounds associated with the aromatherapy, fragrance, perfume, insecticide, insect repelling essential oil, fragrance oil or essential oil extract-granular polymer matrix into the gas phase from the polymer-sorbed phase that has previously been achievable in the prior art using monolithic structures. In one embodiment of this present disclosure, the aromatherapy, fragrance, perfume, insecticide, insect repelling essential oil, fragrance oil or essential oil extract-granular polymer matrix coated particles are placed within a gas permeable, gas semi-permeable or microporous housing, selected from the group containing:
a) gas permeable woven fabrics;
b) gas permeable non-woven fabrics;
c) injected molded gas permeable plastic mesh materials;
d) gas permeable hoses;
e) porous ceramic;
f) porous metal;
g) gas permeable filters;
h) gas permeable filter fabrics;
i) necklace pendants; and
j) gas permeable housings selected from the group consisting of natural and synthetic fabrics consisting of: wool; cotton; bamboo; hemp; nylon; polyester nylon; polyester; acrylic; polypropylene; rayon; polyolefin; and materials sold under the trade name Gore-Tex®; Tyvek®; Evolution®; or Optima™.

In another embodiment of this present disclosure, the aromatherapy, fragrance, perfume, insecticide, insect repelling essential oil, fragrance oil or essential oil extract-granular polymer matrix coated particles are placed in a cylinder, rectangular or cubical housing with solid gas impermeable sides and is open on two ends comprising a filter housing that retains the aromatherapy, fragrance, perfume, insecticide, insect repelling essential oil, fragrance oil or essential oil extract-granular polymer matrix coated particles in the housing by mesh screens or other gas permeable materials that can retain the matrix inside the housing. The filter housing containing the aromatherapy, fragrance, perfume, insecticide, insect repelling essential oil, fragrance oil or essential oil extract-granular polymer matrix coated particles is then connected using gas tight fittings or couplers to the exhaust end of an air blower or air pump. The exhaust end of the filter housing is then connected to a single or multi-directional air diffuser, open pipe or air duct to direct and discharge the volatile organic carbon laden air into an enclosed, semi-enclosed or open space. In another embodiment of the present disclosure, the exhaust end of the filter housing is connected to a manifold or a series of pipes or ductwork that conveys the volatile organic carbon laden air to specific locations within an enclosed, semi-enclosed or open space. A person having ordinary skill in the art would recognize than a multitude of air conveyance and air distribution systems and equipment could be used to convey the volatile organic carbon laden air exhausting from the filter housing attached to an air blower or air pump to specific locations within enclosed, semi-enclosed or open spaces. A manifold system can be adjusted by measuring air flow rates exiting from several air exit locations from a manifold system and by opening or closing baffles, valves, dampers or diffusion blades to either increase or decrease air flow rates to specific locations.

In an embodiment of this disclosure, one or more insect repelling, insecticidal, aromatherapy or fragrance emitting volatile organic compounds with a vapor pressure from about 0.0002 mm Hg at 20° C. to about 40.000 mm Hg at 20° C. are sorbed with a granular polymer at an essential oil, fragrance, perfume, insecticide, insect repelling or essential oil extract volatile organic compound to polymer ratio between about 1.0 to about 15.0 and the aromatherapy, fragrance, perfume, insecticide, insect repelling essential oil, fragrance oil or essential oil extract/granular polymer mixture is used to coat porous or non-porous solid particles made from either natural or man-made materials or man-made absorption or stripping type mass transfer packing material particles at an aromatherapy, fragrance, perfume, insecticide, insect repelling essential oil, fragrance oil or essential oil extract-granular polymer matrix to porous or non-porous solid particle ratio from about 0.25 to about 5.0 and the aromatherapy, fragrance, perfume, insecticide, insect repelling essential oil, fragrance oil or essential oil extract/granular polymer/solid particle matrix is packed into a porous housing at a bulk density varying from about 0.20 g/cm³ to about 2.0 g/cm³. When the solid particle is either a porous or non-porous plastic material, such as polyethylene pellets with particle sizes varying in length, width and depth dimensions, for example from about 1.0 mm to about 5.0 mm, the packed bulk density of the matrix can vary for example from about 0.35 g/cm³ to about 0.75 g/cm³. When the solid particle is a pea stone gravel with a particle sizes varying in length, width and depth dimensions from for example from about 1.0 mm to about 5.0 mm, the packed bulk density of the matrix can vary for example from about 0.8 g/cm³ to about 1.5 g/cm³. When the solid particle is a wood pellet with a particle size varying in length, width and depth dimensions from for example from about 1.5 mm to about 5 mm the packed bulk density of the matrix can vary from about 0.50 g/cm³ to about 0.90 g/cm³. When the porous particle is a man-made absorption or stripping type mass transfer packing material with a particle size varying from about 0.25 inch (6.35 mm) to about 1.5 inch (38.1 mm) the packed density of the aromatherapy, fragrance, perfume, insecticide, insect repelling essential oil, fragrance oil or essential oil extract/granular polymer/solid particle matrix coated or embedded particles is from about 0.25 g/cm³ to about 0.50 g/cm³.

In another embodiment of the present disclosure, the natural or man-made porous or non-porous solid particles have a packed total porosity from about 0.05 to about 0.60.

In yet another embodiment of the present disclosure, the man-made absorption or stripping type mass transfer packing material coated or embedded with the of aromatherapy, fragrance, perfume, insecticide, insect repelling essential oil, fragrance oil or essential oil extract/granular polymer mixture the total porosity can range from about 0.50 to about 0.85.

In yet another embodiment of the present disclosure, the aromatherapy, fragrance, perfume, insecticide, insect repelling essential oil, fragrance oil or essential oil extract/granular polymer/solid particle matrix has an initial packed total porosity from about 0.01 to about 0.55.

In another embodiment of this present disclosure is a composition which can be manufactured under ambient temperature. In yet another embodiment of this present disclosure the ambient temperature of manufacture is from about 5° C. to about 45° C. In yet another embodiment of this present disclosure the disclosure the ambient temperature of manufacture is from about 15° C. to about 30° C.

In yet another embodiment of this present disclosure, the volatile organic compound is from one or more essential oils, selected from the list consisting of: Abies siberica; Allium sativum; Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum; Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia oliveri; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum; Pinus sylvestri; Pogostemon spp.; Pogostemon cablin; Pelargonium reniforme; Pseudotsuga menziensii′ Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella or Zingiber officinale.

In yet another embodiment of this present disclosure, one or more volatile organic compounds are comprised of one or more extracts from an aromatherapy, fragrance, perfume, insecticide, insect repelling essential oil, fragrance oil or essential oil extract that have a vapor pressure from about 0.0002 mm Hg to about 40.000 mm Hg when measured at a temperature of 20° C. and a pressure of 760 mm Hg.

In another embodiment of this present disclosure, the essential oil extract is selected from the group consisting of: bisabolene; borneol; 3-carene; caryophyllene oxide; camphene; camphor; carvacrol; cis-carveol; cedrol; 1,8-cineole; citral; cis-verbenol; citronellal; citronellol; eugenol; farnesol; geranial; geraniol; 4-isopropylbenzenemethanol; menthol; menthone; methyl salicylate; myrcene; limonene; linalool; p-menthane-3,8-diol; neral; nerol; patchoulol; perillaldehyde; perillyl alcohol; a-pinene; b-pinene; α-terpineol; thymol; vanillin; verbenone or vetviverol.

In another embodiment of the present disclosure the volatile organic carbon compound is a synthetic insecticide, insect repelling compound or pesticide.

In yet another embodiment of the present disclosure the volatile organic carbon compound is a synthetic insecticide, insect repelling or pesticide comprised of one or more compounds selected from the group of consisting of: dichlorvos; DEET (N,N-diethyl toluamide); etofenprox, ethyl hexanediol, cis- or trans-allethrin, p-menthane-3,8-diol, methyl jasmonate, 2-undecanone.

In an embodiment of the present disclosure the polymer used to sorb the aromatherapy, fragrance, perfume, insecticide, insect repelling essential oil, fragrance oil or essential oil extract comprises a hydrophobic oil-sorbing polymer or a hydrophobic oil-solidifying polymer.

In yet another embodiment of this present disclosure the granular hydrophobic oil-sorbing polymers or hydrophobic oil-soldifying polymers are selected from the group consisting of: block copolymers, diblock copolymers; triblock copolymers; radial block polymers, multi-block polymers, cross-linked polymers, crosslinked copolymers of hydroxypropyl cellulose acrylate/octadecyl acrylate; cross-linked polyolefin terpolymers; cross-linked octadecylacrylate-co-acrylic acid copolymers; polynorbomene polymers; styrene-ethylene/butylene-sytrene block copolymers; styrene/butadiene block copolymers; polytertiarybutylstyrene; polyacrylonitrile butadiene; polyisoprene (rubber), polyethylene; polypropylene; poly isobutylene; polyethylene terephthalate fibers modified by the adherence of crosslinked polymer coatings and materials and polymers sold under the trade name Organolock® N65; Norosorex®; Enviro-Bond™ 403; Enviro-Bond™ 405; Elastol 1; Elastol 2; Nochars A 610; Nochars A 650; Omni-Zorb #2000; Omni-Zorb # AZ1N; Omni-Zorb # BZ; Omni-Zorb # PZ; Petro-Lock; Clear-Tec Rubberizer®; SPI Oil Bond, Waste-set PS #3200, and Waste-set PS #3400.

In another embodiment of this present disclosure, naturally occurring or man-made porous or non-porous solid particles are used as porous and air permeable supports to coat with the aromatherapy, fragrance, perfume, insecticide, insect repelling essential oil, fragrance oil or essential oil extract-granular polymer matrix.

In yet another embodiment of this present disclosure man-made porous or non-porous solid particles are selected from the group of thermoset or thermoplastic plastic pellets consisting of: acrylonitrile butadiene styrene (ABS); polyoxymethylene (POM); low density polyethylene (LDPE); high density polyethylene (HDPE); nylon; polyether ether ketone (PEEK); polycarbonate (PC); polyethylene (PE); polypropylene (PP); polystyrene (PS); polyvinyl chloride (PVC).

In yet another embodiment of this present disclosure natural or man-made porous or non-porous solid particles are selected from natural materials.

In yet another embodiment of this present disclosure the natural or man-made porous or non-porous solid particles are selected from the group of natural wood pellets; pea-stone gravel; biomass pellets; coffee husk pellets; hemp pellets; and corn cob pellets with particle sizes varying in length, width and depth dimensions from for example from about 1.0 mm to about 5 mm.

In another embodiment of this present disclosure, the housing that supports the aromatherapy, fragrance, perfume, insecticide, insect repelling essential oil, fragrance oil or essential oil extract-granular polymer matrix coated particles is comprised of a gas permeable, a gas semi-permeable or a microporous material on at least one or on more sides of the housing.

In another embodiment of this present disclosure the housing that supports the aromatherapy, fragrance, perfume, insecticide, insect repelling essential oil, fragrance oil or essential oil extract-granular polymer matrix coated particles optionally has one or more sides comprising a gas and/or liquid impermeable material.

In another embodiment of this present disclosure, a gas and liquid impermeable material comprising one or more sides of the housing that supports the essential oil, fragrance, perfume, insecticide, insect repelling or essential oil extract-granular polymer matrix coated particles is selected from the group consisting of: ethylene vinyl alcohol (EVA) polyester laminate; a low-density polyethylene (LDPE) polyester laminate EVA-LDPE polyester laminate; polytetrafluoroethylene (PTFE); high density polyethylene (HDPE); nylon; polyether ether ketone (PEEK); polycarbonate (PC); polyethylene (PE); polypropylene (PP); polystyrene (PS); polyvinyl chloride (PVC); and materials sold under the trade name Evolution®; Compel®; Barrier 450™; and Optima™.

This present disclosure provides compositions and methods for the passive or active variable rate (slow to fast) sustained time-release of aromatherapy, fragrance, perfume, insecticide, insect repelling essential oil, fragrance oil or essential oil extract emitting volatile organic compounds into the air. One embodiment of this present disclosure provides for a method and a device that is manufactured at ambient temperatures used to repels or inactivate insects or other arthropods by the passive, slow, sustained time-release of repelling, insecticidal or fragrance emitting volatile organic compounds into the air containing:
a) a volatile organic compound-granular polymer matrix consisting of one or more volatile organic compounds sorbed with one or more solid polymers;
b) naturally occurring or man-made porous or non-porous solid particles coated with the volatile organic compound-granular polymer matrix;
c) a gas permeable, gas semi-permeable or a microporous material on at least one side of the structure or container that contains the solid particles coated with the volatile organic compound granular-polymer-matrix; and optionally
d) a gas impermeable material on one or more sides of the material, structure or container that contains the solid particles coated with the volatile organic compound-granular polymer matrix.

Another embodiment of this present disclosure provides for a method and a device that is manufactured at ambient temperatures used to repels or inactivate insects or other arthropods by the active and controllable rate of sustained time-release of repelling, insecticidal or fragrance emitting volatile organic compounds into the air containing:
a) a volatile organic compound-granular polymer matrix consisting of one or more volatile organic compounds sorbed with one or more solid polymers;
b) Man-made porous plastic, metal or ceramic mass transfer packing media coated or embedded with the volatile organic compound-granular polymer matrix and placed in a cylindrical, rectangular or cubical filter housing open on at least two ends;
c) An air blower or air pump with the air exhaust that passes through the filter housing and volatilizes the insect repelling, insecticidal or fragrance emitting volatile organic compounds into the air stream; and
d) A pipe or series of pipes or air handling ductwork that conveys and directs the insect repelling, insecticidal or fragrance emitting volatile organic compounds into enclosed, semi-enclosed or open air spaces.

In yet another embodiment of this present disclosure provides for a method and a device that is manufactured at ambient temperatures used to provide an aromatherapy environment passive, slow, sustained time-release of aromatherapy or fragrance emitting volatile organic compounds into the air containing:
a) a volatile organic compound-granular polymer matrix consisting of one or more volatile organic compounds sorbed with one or more solid polymers;
b) naturally occurring or man-made porous or non-porous solid particles coated with the volatile organic compound-granular polymer matrix;
c) a gas permeable, gas semi-permeable or a microporous material on at least one side of the structure or container that contains the solid particles coated with the volatile organic compound granular-polymer-matrix; and optionally
d) a gas impermeable material on one or more sides of the material, structure or container that contains the solid particles coated with the volatile organic compound-granular polymer matrix.

In yet another embodiment of this present disclosure provides for a method and a device that is manufactured at ambient temperatures used to provide an aromatherapy environment by the active and controllable rate of sustained time-release of aromatherapy or fragrance emitting volatile organic compounds into the air containing:
a) a volatile organic compound-granular polymer matrix consisting of one or more volatile organic compounds sorbed with one or more solid polymers;
b) Man-made porous plastic, metal or ceramic mass transfer packing media coated or embedded with the volatile organic compound-granular polymer matrix and placed in a cylindrical, rectangular or cubical filter housing open on at least two ends;
c) An air blower or air pump with the air exhaust that passes through the filter housing and volatilizes the insect repelling, insecticidal or fragrance emitting volatile organic compounds into the air stream; and
d) A pipe or series of pipes or air handling ductwork that conveys and directs the insect repelling, insecticidal or fragrance emitting volatile organic compounds into enclosed, semi-enclosed or open air spaces.

One embodiment of this present disclosure is an improvement from prior art in which aromatherapy, fragrances, perfumes, insecticides, essential oils or essential oil extracts have been made into gels with polymers or sorbed with polymers. In the prior art, the aromatherapy, fragrances, perfumes, insecticides, essential oils or essential oil extracts that have been made into gels or sorbed onto polymers are limited in surface area contact with a gas phase or ambient air and therefore limits the effectiveness of the mass transfer and diffusion of the volatile organic compounds associated with these materials into the gas phase or ambient air. As a result several applications in the prior art require a mechanical device, for example a blower or a heater to accelerate the mass flux of the volatile organic materials into the gas phase to achieve a desired effect. The mass flux of volatile organic compounds into a gas phase or the ambient air is controlled in part by the surface area exposed to a gas phase or the ambient air. In this embodiment of the present disclosure the large surface area associated with porous or non-porous particles coated with the aromatherapy, fragrance, perfume, insecticide, insect repelling essential oil extract-granular polymer matrix enables sufficient diffusion and mass transfer of volatile organic compounds associated with the aromatherapy, fragrance, perfume, insecticide, insect repelling essential oil extract-granular polymer matrix into a gas phase or ambient air passively, without the requirement of a mechanical device such as a heater or blower. Non-Aqueous Phase Liquid organic compounds are defined in this patent as organic compounds that are immiscible in water, have limited solubility in water, are hydrophobic and have a vapor pressure greater than 0.0002 mm Hg at 20° C.

In another embodiment of this present disclosure, coating or embedding of larger size structured or random porous 0.25 inch (6.35 mm) to 1.5 inch (38.1 mm) man-made porous plastic, metal or ceramic mass transfer packing media with the volatile organic compound-granular polymer matrix and placing the coated media in a cylindrical, rectangular or cubical filter housing open on at least two ends provides for low air pressure drop across the filter and efficient mass transfer of the volatile organic compounds from the volatile organic compound-granular polymer matrix into the gas or air phase for effective delivery of the fragrance, perfume, insecticide, insect repelling volatile organic compounds into an enclosed, semi-enclosed or open space. The use of an air blower to air pump to create air flow rates from 1 cubic foot per minute to 10000 cubic feet minute enables larger volume enclosed, semi-enclosed or open spaces to be effectively treated with aromatherapy, fragrance, perfume, insecticide, or insect repelling volatile organic compounds and at higher volatile organic compounds concentrations and at faster rates of emission than is possible with the passive release of aromatherapy fragrance, perfume, insecticide, insect repelling volatile organic compounds from the porous or non-porous particles coated solid particles placed in a passive release container.

In yet another embodiment of this present disclosure is a method where man-made absorption or stripping type mass transfer packing material has a particle size varying from about 0.25 inch (6.35 mm) to about 1.5 inch (38.1 mm) the packed density aromatherapy, fragrance, perfume, insecticide, insect repelling essential oil, fragrance oil or essential oil extract/polymer/solid particle matrix coated or embedded particles is from about 0.25 g/cm³ to about 0.50 g/cm³.

In another embodiment of this present disclosure is a method where the ratio of the aromatherapy, fragrance, perfume, insecticide, insect repelling essential oil, fragrance oil or essential oil extract volatile organic carbon plus granular polymer to solid pellet ratio is from about 0.05 to about 3.00 on a weight basis.

In another embodiment of this present disclosure is a method where the bulk density of the aromatherapy, fragrance, perfume, insecticide, insect repelling essential oil, fragrance oil or essential oil extract volatile organic carbon/granular polymer/solid particle matrix contained within into the porous housing is from about 0.35 g/cm³ to about 3.0 g/cm³.

In another embodiment of this present disclosure is a method where the porous or non-porous solid particles have a packed total porosity from about 0.05 to about 0.60.

In another embodiment of this present disclosure is a method where aromatherapy, fragrance, perfume, insecticide, insect repelling essential oil, fragrance oil or essential oil extract volatile organic carbon/granular polymer/solid particle matrix have an initial packed total porosity from about 0.01 to about 0.55.

In another embodiment of this present disclosure is a method where the ambient temperature of manufacture is from about 5° C. to about 45° C.

In another embodiment of this present disclosure is a method where the device housing the aromatherapy, fragrance, perfume, insecticide, insect repelling essential oil, fragrance oil or essential oil extract volatile organic carbon/granular polymer/solid particle matrix is porous with a porosity from about 0.01 to about 0.75.

In another embodiment of this present disclosure is a method where the device housing the aromatherapy, fragrance, perfume, insecticide, insect repelling essential oil, fragrance oil or essential oil extract volatile organic carbon/granular polymer/solid particle matrix has pore-size dimensions from about 0.005 mm to about 3.00 mm.

In another embodiment of this present disclosure is a method where the device housing the aromatherapy, fragrance, perfume, insecticide, insect repelling essential oil, fragrance oil or essential oil extract volatile organic carbon/granular polymer/solid particle matrix is a gas permeable or a gas semi-permeable fabric or material.

In another embodiment of this present disclosure is a method where the device housing the aromatherapy, fragrance, perfume, insecticide, insect repelling essential oil, fragrance oil or essential oil extract volatile organic carbon/granular polymer/solid particle matrix is injected molded with at least one side being porous.

In another embodiment of this present disclosure is a method where the device housing the aromatherapy, fragrance, perfume, insecticide, insect repelling essential oil, fragrance oil or essential oil extract volatile organic carbon/granular polymer/solid particle matrix is exposed to an enclosed air space and passively releases volatile organic compounds to the air.

In yet another embodiment of this present disclosure is a method where the device housing the aromatherapy, fragrance, perfume, insecticide, insect repelling essential oil, fragrance oil or essential oil extract volatile organic carbon/granular polymer/solid particle matrix is exposed to a semi-enclosed air space and passively releases volatile organic compounds to the air.

In yet another embodiment of this present disclosure is a method where the device housing the aromatherapy, fragrance, perfume, insecticide, insect repelling essential oil, fragrance oil or essential oil extract volatile organic carbon/granular polymer/solid particle matrix is exposed to an open air space and passively releases volatile organic compounds to the air.

In yet another embodiment of this present disclosure is a method where the device housing the aromatherapy, fragrance, perfume, insecticide, insect repelling essential oil, fragrance oil or essential oil extract volatile organic carbon/granular polymer/solid particle matrix is placed in an advectively flowing air stream and releases volatile organic compounds to the air.

In another embodiment of this present disclosure activated carbon, either granular or powdered, is incorporated into the aromatherapy, fragrance, perfume, insecticide, or insect repelling essential oil, fragrance oil, or essential oil extract-granular polymer matrix used by itself without coating porous or non-porous particles or by coating porous or non-porous solid particles.

In yet another embodiment of this present disclosure the aromatherapy, fragrance, perfume, insecticide, or insect repelling essential oil, fragrance oil, or essential oil extract-granular polymer matrix used by itself without coating porous or non-porous particles or by coating porous or non-porous solid particles can be placed in a structure that contains the aromatherapy or fragrance volatile organic compound-granular polymer matrix that can be opened to the atmosphere on one or more sides, as well as a structure that contains the aromatherapy or fragrance volatile organic compound-granular polymer matrix that consists of a gas impermeable material sealed or closed to the atmosphere on one or more sides.

In an embodiment of this present disclosure is a device that is manufactured at ambient temperatures and a method used for aromatherapy or introducing a fragrance by the passive, slow, sustained time-release emissions of aromatherapy or fragrance volatile organic compounds into the air comprising:
a) an aromatherapy or fragrance volatile organic compound-granular polymer matrix consisting of one or more aromatherapy or fragrance volatile organic compounds sorbed with one or more solid polymers;
b) a naturally occurring or man-made porous or non-porous solid particles coated with the aromatherapy or fragrance volatile organic compound-granular polymer matrix;
c) a gas permeable, gas semi-permeable or a microporous material on at least one side of the structure that contains the solid particles coated with the aromatherapy or fragrance volatile organic compound-polymer-solid particle mixture; and optionally
d) a gas impermeable material on one or more sides of the material that contains the solid particles coated with the aromatherapy or fragrance volatile organic compound-granular polymer matrix.

In an embodiment of this present disclosure is a composition and a method, wherein the aromatherapy or fragrance volatile organic compound that provides aromatherapy or fragrance has a vapor pressure from about 0.0002 mm Hg to about 40.000 mm Hg when measured at a temperature of 20° C. and a pressure of 760 mm Hg.

In an embodiment of this present disclosure is a composition and a method where the aromatherapy or fragrance volatile organic compound is an essential oil.

In yet another embodiment of this present disclosure is a composition and a method where the essential oil, fragrance, perfume, insecticide, insect repelling or essential oil extract essential oil is selected from the group consisting of: Abies siberica; Allium sativum; Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum; Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia oliveri; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum; Pinus sylvestri; Pogostemon spp.; Pogostemon cablin; Pelargonium reniforme; Pseudotsuga menziensii′ Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella or Zingiber officinale.

In another embodiment of this present disclosure is a composition and a method where the essential oil is an extract from an essential oil.

In another embodiment of this present disclosure is a composition and a method where the ratio of aromatherapy or fragrance volatile organic carbon to granular polymer sorbent is from about 1 to about 15 on a weight basis.

In yet another embodiment of this present disclosure is a composition and a method where the ratio of the aromatherapy or fragrance volatile organic carbon plus granular polymer solid pellet ratio is from about 0.05 to about 3.00 on a weight basis.

In yet another embodiment of this present disclosure is a composition and a method where the bulk density of the aromatherapy or fragrance volatile organic carbon/granular polymer/solid particle matrix contained within into the porous housing is from about 0.35 g/cm³ to about 2.0 g/cm³.

In yet another embodiment of this present disclosure is a composition and a method where the porous or non-porous solid particles have a packed total porosity from about 0.05 to about 0.60.

In yet another embodiment of this present disclosure is a composition and a method where aromatherapy or fragrance volatile organic carbon/granular polymer/solid particle matrix have an initial packed total porosity from about 0.01 to about 0.55.

In yet another embodiment of this present disclosure is a composition and a method where the ambient temperature of manufacture is from about 5° C. to about 45° C.

In another embodiment of this present disclosure is a method to provide aromatherapy or fragrance by the passive, slow, sustained time-release of aromatherapy or fragrance emitting volatile organic compounds into the air comprising:
a) an aromatherapy or fragrance volatile organic compound-granular polymer matrix consisting of one or more volatile organic compounds sorbed with one or more solid polymers;
b) a naturally occurring or man-made porous or non-porous solid particles coated with the aromatherapy or fragrance volatile organic compound-granular polymer matrix;
c) a gas permeable, gas semi-permeable or a microporous material on at least one side of the structure that contains the solid particles coated with an aromatherapy or fragrance volatile organic compound-polymer-solid particle mixture; and optionally
d) a gas impermeable material on one or more sides of the material that contains the solid particles coated with the aromatherapy or fragrance volatile organic compound-granular polymer matrix.

In another embodiment of this present disclosure is a method wherein the aromatherapy or fragrance volatile organic compound that provides aromatherapy and fragrance has a vapor pressure from about 0.0002 mm Hg to about 40.000 mm Hg when measured at a temperature of 20° C. and a pressure of 760 mm Hg.

Preferred embodiments of this disclosure are described in clauses 1-287 below.

1. A device that is manufactured at ambient temperatures used to repels or inactivate insects or other arthropods by the passive, slow, sustained time-release of repelling, insecticidal or fragrance emitting volatile organic compounds into the air comprising:
a) an essential oil, fragrance, perfume, insecticide, insect repelling or essential oil extract volatile organic compound-granular polymer matrix consisting of one or more volatile organic compounds sorbed with one or more granular polymers;
b) naturally occurring or man-made porous or non-porous solid particles coated with the volatile organic compound-granular polymer matrix;
c) a gas permeable, gas semi-permeable or a microporous material on at least one side of the structure that contains the solid particles coated with the volatile organic compound-polymer-solid particle mixture; and optionally
d) a gas impermeable material on one or more sides of the material that contains the solid particles coated with the volatile organic compound-granular polymer matrix.

2. The device of clause 1 wherein the essential oil, fragrance, perfume, insecticide, insect repelling or essential oil extract volatile organic compound that repels or inactivates insects or other arthropods has a vapor pressure from about 0.0002 mm Hg to about 40.000 mm Hg when measured at a temperature of 20° C. and a pressure of 760 mm Hg.

3. The device of clause 1 where the volatile organic compound is an essential oil.

4. The device of clause 1 wherein the essential oil, fragrance, perfume, insecticide, insect repelling or essential oil extract essential oil is selected from the group consisting of: Abies siberica; Allium sativum; Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum; Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia oliveri; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum; Pinus sylvestri; Pogostemon spp.; Pogostemon cablin; Pelargonium reniforme; Pseudotsuga menziensii; Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella or Zingiber officinale.

5. The device of clause 3 wherein the essential oil is an extract from an essential oil.

6. The device of clause 5 where the essential oil extract is selected from the group consisting of: bisabolene; borneol; 3-carene; caryophyllene oxide; camphene; camphor; carvacrol; cis-carveol; cedrol; 1,8-cineole; citral; cis-verbenol; citronellal; citronellol; eugenol; farnesol; geranial; geraniol; 4-isopropylbenzenemethanol; menthol; menthone; methyl salicylate; myrcene; limonene; linalool; p-menthane-3,8-diol; neral; nerol; patchoulol; perillaldehyde; perillyl alcohol; a-pinene; b-pinene; α-terpineol; thymol; vanillin; verbenone or vetviverol.

7. The device of clause 1 where the insecticidal volatile organic compound is an insecticide.

8. The device of clause 7 where the insecticidal volatile organic compound is selected from the group consisting of: dichlorvos; DEET (N,N-diethyl toluamide); etofenprox; or ethyl hexanediol.

9. The device of clause 1 where the granular polymer is a hydrophobic oil sorbing polymer or a hydrophobic oil solidifying polymer.

10. The device of clause 1 where the hydrophobic oil sorbing polymers include but are not limited to those selected from the group consisting of: block copolymers, diblock copolymers; triblock copolymers; radial block polymers, multi-block polymers, cross-linked polymers, crosslinked copolymers of hydroxypropyl cellulose acrylate/octadecyl acrylate; cross-linked polyolefin terpolymers; cross-linked octadecylacrylate-co-acrylic acid copolymers; polynorbornene polymers; styrene-ethylene/butylene-sytrene block copolymers; styrene/butadiene block copolymers; polytertiarybutylstyrene; polyacrylo-nitrile butadiene; polyisoprene (rubber), polyethylene; polypropylene; poly isobutylene; polyethylene terephthalate fibers modified by the adherence of crosslinked polymer coatings and materials and polymers sold under the trade name Organolock® N65; Norosorex®; Enviro-Bond™ 403; Enviro-Bond™ 405; Elastol 1; Elastol 2; Nochars A 610; Nochars A 650; Omni-Zorb #2000; Omni-Zorb # AZ1N; Omni-Zorb # BZ; Omni-Zorb # PZ; Petro-Lock; Clear-Tec Rubberizer®; SPI Oil Bond, Waste-set PS #3200, and Waste-set PS #3400.

11. The device of clause 1 where the man-made porous or non-porous solid particles are selected from the group of thermoplastic plastic pellets including but not limited to those made with acrylonitrile butadiene styrene (ABS); polyoxymethylene (POM); low density polyethylene (LDPE); high density polyethylene (HDPE); nylon; polyether ether ketone (PEEK); polycarbonate (PC); polyethylene (PE); polypropylene (PP); polystyrene (PS); polyvinyl chloride (PVC).

12. The device of clause 1 where the solid particles are made from natural materials selected from the group of natural wood pellets; pea-stone gravel; biomass pellets; coffee husk pellets; hemp pellets; corn cob pellets.

13. The device of clause 1 where the solid pellets are either a porous or non-porous with particle sizes varying in length, width and depth dimensions from for example from 1.0 mm to 5.0 mm.

14. The device of clause 1 where the gas permeable, gas semi-permeable or microporous with materials are selected from the group consisting of gas permeable woven fabrics, non-woven fabrics, injected molded gas permeable plastic mesh materials, gas permeable housings, selected from the group consisting of natural and synthetic fabrics consisting of: wool; cotton; bamboo; hemp; nylon; polyester nylon; polyester; acrylic; rayon; polyolefin; and materials sold under the trade name Gore-Tex®; Tyvek®; Evolution®; or Optima™.

15. The device of clause 1 where the optional gas impermeable material is selected from the group consisting of ethylene vinyl alcohol (EVA) polyester laminate; a low-density polyethylene (LDPE) polyester laminate EVA-LDPE polyester laminate; polytetrafluoroethylene (PTFE); high density polyethylene (HDPE); nylon; polyether ether ketone (PEEK); polycarbonate (PC); polyethylene (PE); polypropylene (PP); polystyrene (PS); polyvinyl chloride (PVC); and materials sold under the trade name Evolution®; Compel®; Barrier 450™; and Optima™.

16. The device of clause 1 where the ratio of essential oil, fragrance, perfume, insecticide, insect repelling or essential oil extract volatile organic carbon to granular polymer sorbent is from about 1 to about 15 on a weight basis.

17. The device of clause 1 where the ratio of the essential oil, fragrance, perfume, insecticide, insect repelling or essential oil extract volatile organic carbon plus granular polymer to solid pellet ratio is from about 0.05 to about 3.00 on a weight basis.

18. The device of clause 1 where the bulk density of the essential oil, fragrance, perfume, insecticide, insect repelling or essential oil extract volatile organic carbon/granular polymer/solid particle matrix contained within into the porous housing is from about 0.20 g/cm³ to about 2.0 g/cm³.

19. The device of clause 1 where the porous or non-porous solid particles have a packed total porosity from about 0.05 to about 0.60.

20. The device of clause 1 where the essential oil, fragrance, perfume, insecticide, insect repelling or essential oil extract volatile organic carbon/granular polymer/solid particle matrix have an initial packed total porosity from about 0.01 to about 0.55.

21. The device of clause 1 where the ambient temperature of manufacture is from about 5° C. to about 45° C.

22. A method to repel or inactivate insects or other arthropods by the passive, slow, sustained time-release of repelling, insecticidal or fragrance emitting volatile organic compounds into the air comprising:
a) an essential oil, fragrance, perfume, insecticide, insect repelling or essential oil extract volatile organic compound-granular polymer matrix consisting of one or more volatile organic compounds sorbed with one or more solid polymers;
b) naturally occurring or man-made porous or non-porous solid particles coated with the essential oil, fragrance, perfume, insecticide, insect repelling or essential oil extract volatile organic compound-granular polymer matrix;
c) a gas permeable, gas semi-permeable or a microporous material on at least one side of the structure that contains the solid particles coated with an essential oil, fragrance, perfume, insecticide, insect repelling or essential oil extract volatile organic compound-granular polymer-solid particle mixture; and optionally
d) a gas impermeable material on one or more sides of the material that contains the solid particles coated with the essential oil, fragrance, perfume, insecticide, insect repelling or essential oil extract volatile organic compound-granular polymer matrix.

23. The method of clause 22 wherein the essential oil, fragrance, perfume, insecticide, insect repelling or essential oil extract volatile organic compound that repels or inactivates insects or other arthropods has a vapor pressure from about 0.0002 mm Hg to about 40.000 mm Hg when measured at a temperature of 20° C. and a pressure of 760 mm Hg.

24. The method of clause 22 where the essential oil, fragrance, perfume, insecticide, insect repelling or essential oil extract volatile organic compound is an essential oil.

25. The method of clause 24 wherein the essential oil is selected from the group consisting of: Abies siberica; Allium sativum; Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum; Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia oliveri; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum; Pinus sylvestri; Pogostemon spp.; Pogostemon cablin; Pelargonium reniforme; Pseudotsuga menziensii; Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella or Zingiber officinale.

26. The method of clause 22 wherein the essential oil is an extract from an essential oil.

27. The method of clause 22 wherein the essential oil is an extract from an essential oil selected from the group consisting of: bisabolene; borneol; 3-carene; caryophyllene oxide; camphene; camphor; carvacrol; cis-carveol; cedrol; 1,8-cineole; citral; cis-verbenol; citronellal; citronellol; eugenol; farnesol; geranial; geraniol; 4-isopropylbenzenemethanol; menthol; menthone; methyl salicylate; myrcene; limonene; linalool; p-menthane-3,8-diol; neral; nerol; patchoulol; perillaldehyde; perillyl alcohol; a-pinene; b-pinene; α-terpineol; thymol; vanillin; verbenone or vetviverol.

28. The method of clause 22 where the insecticidal volatile organic compound is an insecticide.

29. The method of clause 28 where the insecticidal volatile organic compound is selected from the group of consisting of: dichlorvos; DEET (N,N-diethyl toluamide); etofenprox, and ethyl hexanediol.

30. The method of clause 22 where the granular polymer is a hydrophobic oil sorbing polymer or a hydrophobic oil solidifying polymer.

31. The method of clause 30 where the hydrophobic oil sorbing polymers including but not limited to those selected from the group consisting of: block copolymers, diblock copolymers; triblock copolymers; radial block polymers, multi-block polymers, cross-linked polymers, crosslinked copolymer of hydroxypropyl cellulose acrylate/octadecyl acrylate; cross-linked polyolefin terpolymer; cross-linked octadecylacrylate-co-acrylic acid copolymers; polynorbornene polymer; styrene-ethylene/butylene-sytrene block copolymer; styrene/butadiene block copolymer; polytertiarybutylstyrene; polyacrylo-nitrile butadiene; polyisoprene (rubber), polyethylene; polypropylene; poly isobutylene; polyethylene terephthalate fibers modified by the adherence of crosslinked polymer coatings and materials and polymers sold under the trade name Organolock® N65; Norosorex®; Enviro-Bond™ 403; Enviro-Bond™ 405; Elastol 1; Elastol 2; Nochars A 610; Nochars A 650; Omni-Zorb #2000; Omni-Zorb # AZ1N; Omni-Zorb # BZ; Omni-Zorb # PZ; Petro-Lock; Clear-Tec Rubberizer®; SPI Oil Bond, Waste-set PS #3200, and Waste-set PS #3400.

32. The method of clause 22 where the man-made porous or non-porous solid particles are selected from the group of thermoplastic plastic pellets including but not limited to those made with acrylonitrile butadiene styrene (ABS); polyoxymethylene (POM); low density polyethylene (LDPE); high density polyethylene (HDPE); nylon; polyether ether ketone (PEEK); polycarbonate (PC); polyethylene (PE); polypropylene (PP); polystyrene (PS); polyvinyl chloride (PVC).

33. The method of clause 22 where the solid particles are made from natural materials selected from the group of natural wood pellets; pea-stone gravel; biomass pellets; coffee husk pellets; hemp pellets; corn cob pellets.

34. The method of clause 22 where the solid pellets are either a porous or non-porous with particle sizes varying in length, width and depth dimensions from for example from about 1.0 mm to about 5 mm.

35. The method of clause 22 where the gas permeable, gas semi-permeable or microporous with materials are selected from the group consisting of gas permeable woven fabrics, non-woven fabrics, injected molded gas permeable plastic mesh materials, gas permeable housings, selected from the group consisting of natural and synthetic fabrics consisting of: wool; cotton; bamboo; hemp; nylon; polyester nylon; polyester; acrylic; rayon; polyolefin; and materials sold under the trade name Gore-Tex®; Tyvek®; Evolution®; or Optima™.

36. The method of clause 22 where the optional gas impermeable material is selected from the group consisting of ethylene vinyl alcohol (EVA) polyester laminate; a low-density polyethylene (LDPE) polyester laminate EVA-LDPE polyester laminate; polytetrafluoroethylene (PTFE); high density polyethylene (HDPE); nylon; polyether ether ketone (PEEK); polycarbonate (PC); polyethylene (PE); polypropylene (PP); polystyrene (PS); polyvinyl chloride (PVC); and materials sold under the trade name Evolution®; Compel®; Barrier 450™; and Optima™.

37. The method of clause 22 where the ratio of essential oil, fragrance, perfume, insecticide, insect repelling or essential oil extract volatile organic carbon to granular polymer sorbent is from about 1 to about 15 on a weight basis.

38. The method of clause 22 where the ratio of the essential oil, fragrance, perfume, insecticide, insect repelling or essential oil extract volatile organic carbon plus granular polymer to solid pellet ratio is from about 0.05 to about 3.00 on a weight basis.

39. The method of clause 22 where the bulk density of the essential oil, fragrance, perfume, insecticide, insect repelling or essential oil extract volatile organic carbon/granular polymer/solid particle matrix contained within into the porous housing is from about 0.20 g/cm³ to about 3.0 g/cm³.

40. The method of clause 22 where the porous or non-porous solid particles have a packed total porosity from about 0.05 to about 0.60

41. The method of clause 22 where the essential oil, fragrance, perfume, insecticide, insect repelling or essential oil extract volatile organic carbon/granular polymer/solid particle matrix have an initial packed total porosity from about 0.01 to about 0.55.

42. The method of clause 22 where the ambient temperature of manufacture is from about 5° C. to about 45° C.

43. The method of clause 22 where the device housing the essential oil, fragrance, perfume, insecticide, insect repelling or essential oil extract volatile organic carbon/granular polymer/solid particle matrix is porous with a porosity from about 0.01 to about 0.75.

44. The method of clause 22 where the device housing the essential oil, fragrance, perfume, insecticide, insect repelling or essential oil extract volatile organic carbon/granular polymer/solid particle matrix has pore-size dimensions from about 0.005 mm to about 3.00 mm.

45. The method of clause 22 where the device housing the essential oil, fragrance, perfume, insecticide, insect repelling or essential oil extract volatile organic carbon/granular polymer/solid particle matrix is a gas permeable or a gas semi-permeable fabric or material.

46. The method of clause 22 where the device housing the essential oil, fragrance, perfume, insecticide, insect repelling or essential oil extract volatile organic carbon/granular polymer/solid particle matrix is injected molded with at least one side being porous.

47. The method of clause 22 where the device housing the essential oil, fragrance, perfume, insecticide, insect repelling or essential oil extract volatile organic carbon/granular polymer/solid particle matrix is exposed to an enclosed air space and passively releases volatile organic compounds to the air.

48. The method of clause 22 where the device housing the essential oil, fragrance, perfume, insecticide, insect repelling or essential oil extract volatile organic carbon/granular polymer/solid particle matrix is exposed to a semi-enclosed air space and passively releases volatile organic compounds to the air.

49. The method of clause 22 where the device housing the essential oil, fragrance, perfume, insecticide, insect repelling or essential oil extract volatile organic carbon/granular polymer/solid particle matrix is exposed to an open air space and passively releases volatile organic compounds to the air.

50. The method of clause 22 where the device housing the essential oil, fragrance, perfume, insecticide, insect repelling or essential oil extract volatile organic carbon/granular polymer/solid particle matrix is placed in an advectively flowing air stream and releases volatile organic compounds to the air.

51. A device that is manufactured at ambient temperatures used for aromatherapy or introducing a fragrance by the passive, slow, sustained time-release emissions of aromatherapy or fragrance volatile organic compounds into the air comprising:
a) an aromatherapy or fragrance volatile organic compound-granular polymer matrix consisting of one or more aromatherapy or fragrance volatile organic compounds sorbed with one or more solid polymers;
b) naturally occurring or man-made porous or non-porous solid particles coated with the aromatherapy or fragrance volatile organic compound-granular polymer matrix;
c) a gas permeable, gas semi-permeable or a microporous material on at least one side of the structure that contains the solid particles coated with the aromatherapy or fragrance volatile organic compound-polymer-solid particle mixture; and optionally
d) a gas impermeable material on one or more sides of the material that contains the solid particles coated with the aromatherapy or fragrance volatile organic compound-granular polymer matrix.

52. The device of clause 51 wherein the aromatherapy or fragrance volatile organic compound that provides aromatherapy or fragrance has a vapor pressure from about 0.0002 mm Hg to about 40.000 mm Hg when measured at a temperature of 20° C. and a pressure of 760 mm Hg.

53. The device of clause 51 where the aromatherapy or fragrance volatile organic compound is an essential oil.

54. The device of clause 53 wherein the essential oil, fragrance, perfume, insecticide, insect repelling or essential oil extract essential oil is selected from the group consisting of: Abies siberica; Allium sativum; Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum; Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia oliveri; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum; Pinus sylvestri; Pogostemon spp.; Pogostemon cablin; Pelargonium reniforme; Pseudotsuga menziensii; Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella or Zingiber officinale.

55. The device of clause 53 wherein the essential oil is an extract from an essential oil.

56. The device of clause 55 where the essential oil extract is selected from the group consisting of: bisabolene; borneol; 3-carene; caryophyllene oxide; camphene; camphor; carvacrol; cis-carveol; cedrol; 1,8-cineole; citral; cis-verbenol; citronellal; citronellol; eugenol; farnesol; geranial; geraniol; 4-isopropylbenzenemethanol; menthol; menthone; methyl salicylate; myrcene; limonene; linalool; p-menthane-3,8-diol; neral; nerol; patchoulol; perillaldehyde; perillyl alcohol; a-pinene; b-pinene; α-terpineol; thymol; vanillin; verbenone or vetviverol.

57. The device of clause 51 where the granular polymer is a hydrophobic oil sorbing polymer or a hydrophobic oil solidifying polymer.

58. The device of clause 57 where the hydrophobic oil sorbing polymers include but are not limited to those selected from the group consisting of: block copolymers, diblock copolymers; triblock copolymers; radial block polymers, multi-block polymers, cross-linked polymers, crosslinked copolymers of hydroxypropyl cellulose acrylate/octadecyl acrylate; cross-linked polyolefin terpolymers; cross-linked octadecylacrylate-co-acrylic acid copolymers; polynorbornene polymers; styrene-ethylene/butylene-sytrene block copolymers; styrene/butadiene block copolymers; polytertiarybutylstyrene; polyacrylo-nitrile butadiene; polyisoprene (rubber), polyethylene; polypropylene; poly isobutylene; polyethylene terephthalate fibers modified by the adherence of crosslinked polymer coatings and materials and polymers sold under the trade name Organolock® N65; Norosorex®; Enviro-Bond™ 403; Enviro-Bond™ 405; Elastol 1; Elastol 2; Nochars A 610; Nochars A 650; Omni-Zorb #2000; Omni-Zorb # AZ1N; Omni-Zorb # BZ; Omni-Zorb # PZ; Petro-Lock; Clear-Tec Rubberizer®; SPI Oil Bond, Waste-set PS #3200, and Waste-set PS #3400.

59. The device of clause 51 where the man-made porous or non-porous solid particles are selected from the group of thermoplastic plastic pellets including but not limited to those made with acrylonitrile butadiene styrene (ABS); polyoxymethylene (POM); low density polyethylene (LDPE); high density polyethylene (HDPE); nylon; polyether ether ketone (PEEK); polycarbonate (PC); polyethylene (PE); polypropylene (PP); polystyrene (PS); polyvinyl chloride (PVC).

60. The device of clause 51 where the solid particles are made from natural materials selected from the group of natural wood pellets; pea-stone gravel; biomass pellets; coffee husk pellets; hemp pellets; corn cob pellets.

61. The device of clause 51 where the solid pellets are either a porous or non-porous with particle sizes varying in length, width and depth dimensions from for example from 1.0 mm to 5.0 mm.

62. The device of clause 51 where the gas permeable, gas semi-permeable or microporous with materials are selected from the group consisting of gas permeable woven fabrics, non-woven fabrics, injected molded gas permeable plastic mesh materials, gas permeable housings, selected from the group consisting of natural and synthetic fabrics consisting of: wool; cotton; bamboo; hemp; nylon; polyester nylon; polyester; acrylic; rayon; polyolefin; and materials sold under the trade name Gore-Tex®; Tyvek®; Evolution®; or Optima™.

63. The device of clause 51 where the optional gas impermeable material is selected from the group consisting of ethylene vinyl alcohol (EVA) polyester laminate; a low-density polyethylene (LDPE) polyester laminate EVA-LDPE polyester laminate; polytetrafluoroethylene (PTFE); high density polyethylene (HDPE); nylon; polyether ether ketone (PEEK); polycarbonate (PC); polyethylene (PE); polypropylene (PP); polystyrene (PS); polyvinyl chloride (PVC); and materials sold under the trade name Evolution®; Compel®; Barrier 450™; and Optima™.

64. The device of clause 51 where the ratio of aromatherapy or fragrance volatile organic carbon to granular polymer sorbent is from about 1 to about 15 on a weight basis.

65. The device of clause 51 where the ratio of the aromatherapy or fragrance volatile organic carbon plus granular polymer solid pellet ratio is from about 0.05 to about 3.00 on a weight basis.

66. The device of clause 51 where the bulk density of the aromatherapy or fragrance volatile organic carbon/granular polymer/solid particle matrix contained within into the porous housing is from about 0.20 g/cm³ to about 2.0 g/cm³.

67. The device of clause 51 where the porous or non-porous solid particles have a packed total porosity from about 0.05 to about 0.60.

68. The device of clause 51 where aromatherapy or fragrance volatile organic carbon/granular polymer/solid particle matrix have an initial packed total porosity from about 0.01 to about 0.55.

69. The device of clause 51 where the ambient temperature of manufacture is from about 5° C. to about 45° C.

70. A method to provide aromatherapy or fragrance by the passive, slow, sustained time-release of aromatherapy or fragrance emitting volatile organic compounds into the air comprising:
a) an aromatherapy or fragrance volatile organic compound-granular polymer matrix consisting of one or more volatile organic compounds sorbed with one or more solid polymers;
b) naturally occurring or man-made porous or non-porous solid particles coated with the aromatherapy or fragrance volatile organic compound-granular polymer matrix;
c) a gas permeable, gas semi-permeable or a microporous material on at least one side of the structure that contains the solid particles coated with an aromatherapy or fragrance volatile organic compound-polymer-solid particle mixture; and optionally
d) a gas impermeable material on one or more sides of the material that contains the solid particles coated with the aromatherapy or fragrance volatile organic compound-granular polymer matrix.

71. The method of clause 70 wherein the aromatherapy or fragrance volatile organic compound that provides aromatherapy and fragrance has a vapor pressure from about 0.0002 mm Hg to about 40.000 mm Hg when measured at a temperature of 20° C. and a pressure of 760 mm Hg.

72. The method of clause 70 where the aromatherapy or fragrance volatile organic compound is an essential oil.

73. The method of clause 72 wherein the essential oil is selected from the group consisting of: Abies siberica; Allium sativum; Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum; Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia oliveri; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum; Pinus sylvestri; Pogostemon spp.; Pogostemon cablin; Pelargonium reniforme; Pseudotsuga menziensii; Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella or Zingiber officinale.

74. The method of clause 70 where the aromatherapy or fragrance essential oil is an extract from an essential oil.

75. The method of clause 74 wherein the essential oil is an extract from an essential oil selected from the group consisting of: bisabolene; borneol; 3-carene; caryophyllene oxide; camphene; camphor; carvacrol; cis-carveol; cedrol; 1,8-cineole; citral; cis-verbenol; citronellal; citronellol; eugenol; farnesol; geranial; geraniol; 4-isopropylbenzenemethanol; menthol; menthone; methyl salicylate; myrcene; limonene; linalool; p-menthane-3,8-diol; neral; nerol; patchoulol; perillaldehyde; perillyl alcohol; a-pinene; b-pinene; α-terpineol; thymol; vanillin; verbenone or vetviverol.

76. The method of clause 70 where the granular polymer is a hydrophobic oil sorbing polymer or a hydrophobic oil solidifying polymer.

77. The method of clause 76 where the hydrophobic oil sorbing polymers including but not limited to those selected from the group consisting of: block copolymers, diblock copolymers; triblock copolymers; radial block polymers, multi-block polymers, cross-linked polymers, crosslinked copolymer of hydroxypropyl cellulose acrylate/octadecyl acrylate; cross-linked polyolefin terpolymer; cross-linked octadecylacrylate-co-acrylic acid copolymers; polynorbornene polymer; styrene-ethylene/butylene-sytrene block copolymer; styrene/butadiene block copolymer; polytertiarybutylstyrene; polyacrylo-nitrile butadiene; polyisoprene (rubber), polyethylene; polypropylene; poly isobutylene; polyethylene terephthalate fibers modified by the adherence of crosslinked polymer coatings and materials and polymers sold under the trade name Organolock® N65; Norosorex®; Enviro-Bond™ 403; Enviro-Bond™ 405; Elastol 1; Elastol 2; Nochars A 610; Nochars A 650; Omni-Zorb #2000; Omni-Zorb # AZ1N; Omni-Zorb # BZ; Omni-Zorb # PZ; Petro-Lock; Clear-Tec Rubberizer®; SPI Oil Bond, Waste-set PS #3200, and Waste-set PS #3400.

78. The method of clause 70 where the man-made porous or non-porous solid particles are selected from the group of thermoplastic plastic pellets including but not limited to those made with acrylonitrile butadiene styrene (ABS); polyoxymethylene (POM); low density polyethylene (LDPE); high density polyethylene (HDPE); nylon; polyether ether ketone (PEEK); polycarbonate (PC); polyethylene (PE); polypropylene (PP); polystyrene (PS); polyvinyl chloride (PVC).

79. The method of clause 70 where the solid particles are made from natural materials selected from the group of natural wood pellets; pea-stone gravel; biomass pellets; coffee husk pellets; hemp pellets; corn cob pellets.

80. The method of clause 70 where the solid pellets are either a porous or non-porous with particle sizes varying in length, width and depth dimensions from for example from about 1.0 mm to about 5 mm.

81. The method of clause 70 where the gas permeable, gas semi-permeable or microporous with materials are selected from the group consisting of gas permeable woven fabrics, non-woven fabrics, injected molded gas permeable plastic mesh materials, gas permeable housings, selected from the group consisting of natural and synthetic fabrics consisting of: wool; cotton; bamboo; hemp; nylon; polyester nylon; polyester; acrylic; rayon; polyolefin; and materials sold under the trade name Gore-Tex®; Tyvek®; Evolution®; or Optima™.

82. The method of clause 70 where the optional gas impermeable material is selected from the group consisting of ethylene vinyl alcohol (EVA) polyester laminate; a low-density polyethylene (LDPE) polyester laminate EVA-LDPE polyester laminate; polytetrafluoroethylene (PTFE); high density polyethylene (HDPE); nylon; polyether ether ketone (PEEK); polycarbonate (PC); polyethylene (PE); polypropylene (PP); polystyrene (PS); polyvinyl chloride (PVC); and materials sold under the trade name Evolution®; Compel®; Barrier 450™; and Optima™.

83. The method of clause 70 where the ratio of aromatherapy or fragrance volatile organic carbon to granular polymer sorbent is from about 1 to about 15 on a weight basis.

84. The method of clause 70 where the ratio of the aromatherapy or fragrance volatile organic carbon plus granular polymer to solid pellet ratio is from about 0.05 to about 3.00 on a weight basis.

85. The method of clause 70 where the bulk density of the aromatherapy or fragrance volatile organic carbon/granular polymer/solid particle matrix contained within into the porous housing is from about 0.20 g/cm³ to about 3.0 g/cm³.

86. The method of clause 70 where the porous or non-porous solid particles have a packed total porosity from about 0.05 to about 0.60.

87. The method of clause 70 where the aromatherapy or fragrance volatile organic carbon/granular polymer/solid particle matrix have an initial packed total porosity from about 0.01 to about 0.55.

88. The method of clause 70 where the ambient temperature of manufacture is from about 5° C. to about 45° C.

89. The method of clause 70 where the device housing the aromatherapy or fragrance volatile organic carbon/granule polymer/solid particle matrix is porous with a porosity from about 0.01 to about 0.75.

90. The method of clause 70 where the device housing the aromatherapy or fragrance volatile organic carbon/polymer/solid particle matrix has pore-size dimensions from about 0.005 mm to about 3.00 mm.

91. The method of clause 70 where the device housing the aromatherapy or fragrance volatile organic carbon/granular polymer/solid particle matrix is a gas permeable or a gas semi-permeable fabric or material.

92. The method of clause 70 where the device housing the aromatherapy or fragrance volatile organic carbon/granular polymer/solid particle matrix is injected molded with at least one side being porous.

93. The method of clause 70 where the device housing the aromatherapy or fragrance volatile organic carbon/granular polymer/solid particle matrix is exposed to an enclosed air space and passively releases volatile organic compounds to the air.

94. The method of clause 70 where the device housing the aromatherapy or fragrance volatile organic carbon/granular polymer/solid particle matrix is exposed to a semi-enclosed air space and passively releases volatile organic compounds to the air.

95. The method of clause 70 where the device housing the aromatherapy or fragrance volatile organic carbon/granular polymer/solid particle matrix is exposed to an open air space and passively releases volatile organic compounds to the air.

96. The method of clause 70 where the device housing the aromatherapy or fragrance volatile organic carbon/granular polymer/solid particle matrix is placed in an advectively flowing air stream and releases volatile organic compounds to the air.

97. A device that is manufactured at ambient temperatures used to repels or inactivate insects or other arthropods by the active pumping of air through an air filter for the controlled, sustained time-release of repelling, insecticidal or fragrance emitting volatile organic compounds into the air comprising:
a) an essential oil, fragrance, perfume, insecticide, insect repelling or essential oil extract volatile organic compound-granular polymer matrix consisting of one or more volatile organic compounds sorbed with one or more solid polymers;
b) naturally occurring or man-made porous or non-porous solid particles coated with the volatile organic compound-granular polymer matrix;
c) a gas permeable material on at least two sides of a filter housing that contains the solid particles coated with the volatile organic compound-polymer-solid particle mixture; and optionally
d) a gas impermeable material on one or more sides of the filter housing that contains the solid particles coated with the volatile organic compound-granular polymer matrix.

98. The device of clause 97 wherein the essential oil, fragrance, perfume, insecticide, insect repelling or essential oil extract volatile organic compound that repels or inactivates insects or other arthropods has a vapor pressure from about 0.0002 mm Hg to about 40.000 mm Hg when measured at a temperature of 20° C. and a pressure of 760 mm Hg.

99. The device of clause 97 where the volatile organic compound is an essential oil.

100. The device of clause 97 wherein the essential oil, fragrance, perfume, insecticide, insect repelling or essential oil extract essential oil is selected from the group consisting of: Abies siberica; Allium sativum; Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum; Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia oliveri; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum; Pinus sylvestri; Pogostemon spp.; Pogostemon cablin; Pelargonium reniforme; Pseudotsuga menziensii′ Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella or Zingiber officinale.

101. The device of clause 97 wherein the essential oil is an extract from an essential oil.

102. The device of clause 97 where the essential oil extract is selected from the group consisting of: bisabolene; borneol; 3-carene; caryophyllene oxide; camphene; camphor; carvacrol; cis-carveol; cedrol; 1,8-cineole; citral; cis-verbenol; citronellal; citronellol; eugenol; farnesol; geranial; geraniol; 4-isopropylbenzenemethanol; menthol; menthone; methyl salicylate; myrcene; limonene; linalool; p-menthane-3,8-diol; neral; nerol; patchoulol; perillaldehyde; perillyl alcohol; a-pinene; b-pinene; α-terpineol; thymol; vanillin; verbenone or vetviverol.

103. The device of clause 97 where the insecticidal volatile organic compound is an insecticide.

104. The device of clause 103 where the insecticidal volatile organic compound is selected from the group consisting of: dichlorvos; DEET (N,N-diethyl toluamide); etofenprox; or ethyl hexanediol.

105. The device of clause 97 where the granular polymer is a hydrophobic oil sorbing polymer or a hydrophobic oil solidifying polymer.

106. The device of clause 105 where the hydrophobic oil sorbing polymers include but are not limited to those selected from the group consisting of: block copolymers, diblock copolymers; triblock copolymers; radial block polymers, multi-block polymers, cross-linked polymers, crosslinked copolymers of hydroxypropyl cellulose acrylate/octadecyl acrylate; cross-linked polyolefin terpolymers; cross-linked octadecylacrylate-co-acrylic acid copolymers; polynorbornene polymers; styrene-ethylene/butylene-sytrene block copolymers; styrene/butadiene block copolymers; polytertiarybutylstyrene; polyacrylo-nitrile butadiene; polyisoprene (rubber), polyethylene; polypropylene; poly isobutylene; polyethylene terephthalate fibers modified by the adherence of crosslinked polymer coatings and materials and polymers sold under the trade name Organolock® N65; Norosorex®; Enviro-Bond™ 403; Enviro-Bond™ 405; Elastol 1; Elastol 2; Nochars A 610; Nochars A 650; Omni-Zorb #2000; Omni-Zorb # AZ1N; Omni-Zorb # BZ; Omni-Zorb # PZ; Petro-Lock; Clear-Tec Rubberizer®; SPI Oil Bond, Waste-set PS #3200, and Waste-set PS #3400.

107. The device of clause 97 where the man-made porous solid particles are selected from the group of thermoplastic plastic pellets including but not limited to those made with acrylonitrile butadiene styrene (ABS); polyoxymethylene (POM); low density polyethylene (LDPE); high density polyethylene (HDPE); nylon; polyether ether ketone (PEEK); polycarbonate (PC); polyethylene (PE); polypropylene (PP); polystyrene (PS); polyvinyl chloride (PVC).

108. The device of clause 97 where the porous solid particles are absorption or stripping or biofilm support type mass transfer packing materials are porous plastic, metal or ceramic and are commonly and commercially available, for example but not limited to Flexiring™, Intalox™ Snowflake, Cascade Mini-Rings™, BetaRings™ manufactured by Koch-Glitsch LP, Jaeger tri-Paks® manufactured by Rashig USA, Inc., Coralife® Bio-Balls, or Marineland Bio-Filter Balls.

109. The device of clause 108 where the porous solid particles are structured or random porous materials with a nominal size from about 0.25 inch (6.35 mm) to about 1.5 inch (38.1 mm).

110. The device of clause 108 where the porous solid 0.25 inch (6.35 mm) to about 1.5 inch (38.1 mm) particles coated or embedded with the essential oil, fragrance, perfume, insecticide, insect repelling essential oil/granular polymer matrix are packed into a filter support housing or container that allows air to be pumped through the filter with an air blower, an enclosed ring air blower or an air pump, to volatilize the volatile organic compounds from the essential oil, fragrance, perfume, insecticide, insect repelling or essential oil extract volatile organic compound-granular polymer matrix into the air inside the filter housing and then to pump the vapor phase volatile organic compounds into an enclosed space, a semi-enclosed space or an open space.

111. The device of clause 97 where the optional gas impermeable material is selected from the group consisting of sheet metal, stainless steel, galvanized steel, ethylene vinyl alcohol (EVA) polyester laminate; a low-density polyethylene (LDPE) polyester laminate EVA-LDPE polyester laminate; polytetrafluoroethylene (PTFE); high density polyethylene (HDPE); nylon; polyether ether ketone (PEEK); polycarbonate (PC); polyethylene (PE); polypropylene (PP); polystyrene (PS); polyvinyl chloride (PVC); and materials sold under the trade name Evolution®; Compel®; Barrier 450™; Optima™.

112. The device of clause 97 where the ratio of essential oil, fragrance, perfume, insecticide, insect repelling or essential oil extract volatile organic carbon to granular polymer sorbent is from about 1 to about 15 on a weight basis.

113. The device of clause 97 where the ratio of the essential oil, fragrance, perfume, insecticide, insect repelling or essential oil extract volatile organic carbon plus granular polymer to solid pellet ratio is from about 0.05 to about 3.00 on a weight basis.

114. The device of clause 97 where the bulk density of the essential oil, fragrance, perfume, insecticide, insect repelling or essential oil extract volatile organic carbon/granular polymer/solid particle matrix contained within into the porous housing is from about 0.20 g/cm³ to about 2.0 g/cm³.

115. The device of clause 97 where the porous solid particles have a packed total porosity from about 0.50 to about 0.97.

116. The device of clause 97 where the essential oil, fragrance, perfume, insecticide, insect repelling or essential oil extract volatile organic carbon/granular polymer/solid particle matrix have an initial packed total porosity from about 0.20 to about 0.90.

117. The device of clause 97 where the ambient temperature of manufacture is from about 5° C. to about 45° C.

118. A device that is manufactured at ambient temperatures used for aromatherapy or introducing a fragrance by the active pumping of air through an air filter for the controlled, sustained time-release of repelling, insecticidal or fragrance emitting volatile organic compounds into the air comprising:
a) an essential oil, fragrance, perfume or essential oil extract volatile organic compound-granular polymer matrix consisting of one or more volatile organic compounds sorbed with one or more solid polymers;
b) naturally occurring or man-made porous or non-porous solid particles coated with the volatile organic compound-granular polymer matrix;
c) a gas permeable material on at least two sides of a filter housing that contains the solid particles coated with the volatile organic compound-polymer-solid particle mixture; and optionally
d) a gas impermeable material on one or more sides of the filter housing that contains the solid particles coated with the volatile organic compound-granular polymer matrix.

119. The device of clause 118 wherein the essential oil, fragrance, perfume, or essential oil extract volatile organic compound has a vapor pressure from about 0.0002 mm Hg to about 40.000 mm Hg when measured at a temperature of 20° C. and a pressure of 760 mm Hg.

120. The device of clause 118 where the volatile organic compound is an essential oil.

121. The device of clause 118 wherein the essential oil, fragrance, perfume, or essential oil extract essential oil is selected from the group consisting of: Abies siberica; Allium sativum; Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum; Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia oliveri; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum; Pinus sylvestri; Pogostemon spp.; Pogostemon cablin; Pelargonium reniforme; Pseudotsuga menziensii′ Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella or Zingiber officinale.

122. The device of clause 118 wherein the essential oil is an extract from an essential oil.

123. The device of clause 118 where the essential oil extract is selected from the group consisting of: bisabolene; borneol; 3-carene; caryophyllene oxide; camphene; camphor; carvacrol; cis-carveol; cedrol; 1,8-cineole; citral; cis-verbenol; citronellal; citronellol; eugenol; farnesol; geranial; geraniol; 4-isopropylbenzenemethanol; menthol; menthone; methyl salicylate; myrcene; limonene; linalool; p-menthane-3,8-diol; neral; nerol; patchoulol; perillaldehyde; perillyl alcohol; a-pinene; b-pinene; α-terpineol; thymol; vanillin; verbenone or vetviverol.

124. The device of clause 118 where the fragrance is a fragrance oil.

125. The device of clause 118 where the fragrance is a fragrance oil extract.

126. The device of clause 118 where the polymer is a hydrophobic oil sorbing polymer or a hydrophobic oil solidifying polymer.

127. The device of clause 126 where the hydrophobic oil sorbing polymers include but are not limited to those selected from the group consisting of: block copolymers, diblock copolymers; triblock copolymers; radial block polymers, multi-block polymers, cross-linked polymers, crosslinked copolymers of hydroxypropyl cellulose acrylate/octadecyl acrylate; cross-linked polyolefin terpolymers; cross-linked octadecylacrylate-co-acrylic acid copolymers; polynorbornene polymers; styrene-ethylene/butylene-sytrene block copolymers; styrene/butadiene block copolymers; polytertiarybutylstyrene; polyacrylo-nitrile butadiene; polyisoprene (rubber), polyethylene; polypropylene; poly isobutylene; polyethylene terephthalate fibers modified by the adherence of crosslinked polymer coatings and materials and polymers sold under the trade name Organolock® N65; Norosorex®; Enviro-Bond™ 403; Enviro-Bond™ 405; Elastol 1; Elastol 2; Nochars A 610; Nochars A 650; Omni-Zorb #2000; Omni-Zorb # AZ1N; Omni-Zorb # BZ; Omni-Zorb # PZ; Petro-Lock; Clear-Tec Rubberizer®; SPI Oil Bond, Waste-set PS #3200, and Waste-set PS #3400.

128. The device of clause 118 where the man-made porous solid particles are selected from the group of thermoplastic plastic pellets including but not limited to those made with acrylonitrile butadiene styrene (ABS); polyoxymethylene (POM); low density polyethylene (LDPE); high density polyethylene (HDPE); nylon; polyether ether ketone (PEEK); polycarbonate (PC); polyethylene (PE); polypropylene (PP); polystyrene (PS); polyvinyl chloride (PVC).

129. The device of clause 118 where the porous solid particles are absorption or stripping or biofilm support type mass transfer packing materials are porous plastic, metal or ceramic and are commonly and commercially available, for example but not limited to Flexiring™, Intalox™ Snowflake, Cascade Mini-Rings™, BetaRings™ manufactured by Koch-Glitsch LP, Jaeger tri-Paks® manufacture red by Rashig USA, Inc., Coralife® Bio-Balls, or Marineland Bio-Filter Balls.

130. The device of clause 129 where the porous solid particles are structured or random porous materials with a nominal size from about 0.25 inch (6.35 mm) to about 1.5 inch (38.1 mm).

131. The device of clause 118 where the porous solid 0.25 inch (6.35 mm) to about 1.5 inch (38.1 mm) particles coated or embedded with the essential oil, fragrance or essential oil extract/granular polymer matrix are packed into a filter support housing or container that allows air to be pumped through the filter with an air blower, an enclosed ring air blower or an air pump, to volatilize the volatile organic compounds from the essential oil, fragrance, perfume, insecticide, insect repelling or essential oil extract volatile organic compound-granular polymer matrix into the air inside the filter housing and then to pump the vapor phase volatile organic compounds into an enclosed space, a semi-enclosed space or an open space.

132. The device of clause 118 where the optional gas impermeable material is selected from the group consisting of ethylene vinyl alcohol (EVA) polyester laminate; a low-density polyethylene (LDPE) polyester laminate EVA-LDPE polyester laminate; polytetrafluoroethylene (PTFE); high density polyethylene (HDPE); nylon; polyether ether ketone (PEEK); polycarbonate (PC); polyethylene (PE); polypropylene (PP); polystyrene (PS); polyvinyl chloride (PVC); and materials sold under the trade name Evolution®; Compel®; Barrier 450™; and Optima™.

133. The device of clause 118 where the ratio of essential oil, fragrance, perfume or essential oil extract volatile organic carbon to granular polymer sorbent is from about 1 to about 15 on a weight basis.

134. The device of clause 118 where the ratio of the essential oil, fragrance, perfume or essential oil extract volatile organic carbon plus granular polymer to solid pellet ratio is from about 0.05 to about 3.00 on a weight basis.

135. The device of clause 118 where the bulk density of the essential oil, fragrance, perfume or essential oil extract volatile organic carbon/granular polymer/solid particle matrix contained within into the porous housing is from about 0.20 g/cm³ to about 2.0 g/cm³.

136. The device of clause 118 where the porous solid particles have a packed total porosity from about 0.50 to about 0.97.

137. The device of clause 118 where the essential oil, fragrance, perfume or essential oil extract volatile organic carbon/granular polymer/solid particle matrix have an initial packed total porosity from about 0.20 to about 0.90.

138. The device of clause 118 where the ambient temperature of manufacture is from about 5° C. to about 45° C.

139. A method to repel or inactivate insects or other arthropods by the active pumping of air through an air filter for the controlled, sustained time-release of repelling, insecticidal or fragrance emitting volatile organic compounds into the air comprising:
a) an essential oil, fragrance, perfume, insecticide, insect repelling or essential oil extract volatile organic compound-granular polymer matrix consisting of one or more volatile organic compounds sorbed with one or more solid polymers;
b) naturally occurring or man-made porous or non-porous solid particles coated with the volatile organic compound-granular polymer matrix;
c) a gas permeable material on at least two sides of a filter housing that contains the solid particles coated with the volatile organic compound-polymer-solid particle mixture; and optionally
d) a gas impermeable material on one or more sides of the filter housing that contains the solid particles coated with the volatile organic compound-granular polymer matrix.

140. The method of clause 139 wherein the essential oil, fragrance, perfume, insecticide, insect repelling or essential oil extract volatile organic compound that repels or inactivates insects or other arthropods has a vapor pressure from about 0.0002 mm Hg to about 40.000 mm Hg when measured at a temperature of 20° C. and a pressure of 760 mm Hg.

141. The method of clause 139 wherein the volatile organic compound is an essential oil.

142. The method of clause 139 wherein the essential oil, fragrance, perfume, insecticide, insect repelling or essential oil extract essential oil is selected from the group consisting of: Abies siberica; Allium sativum; Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum; Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia oliveri; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum; Pinus sylvestri; Pogostemon spp.; Pogostemon cablin; Pelargonium reniforme; Pseudotsuga menziensii; Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella or Zingiber officinale.

143. The method of clause 139 wherein the essential oil is an extract from an essential oil.

144. The method of clause 139 where the essential oil extract is selected from the group consisting of: bisabolene; borneol; 3-carene; caryophyllene oxide; camphene; camphor; carvacrol; cis-carveol; cedrol; 1,8-cineole; citral; cis-verbenol; citronellal; citronellol; eugenol; farnesol; geranial; geraniol; 4-isopropylbenzenemethanol; menthol; menthone; methyl salicylate; myrcene; limonene; linalool; p-menthane-3,8-diol; neral; nerol; patchoulol; perillaldehyde; perillyl alcohol; a-pinene; b-pinene; α-terpineol; thymol; vanillin; verbenone or vetviverol.

145. The method of clause 139 where the insecticidal volatile organic compound is an insecticide.

146. The method of clause 145 where the insecticidal volatile organic compound is selected from the group consisting of: dichlorvos; DEET (N,N-diethyl toluamide); etofenprox; or ethyl hexanediol.

147. The method of clause 139 where the polymer is a hydrophobic oil sorbing polymer or a hydrophobic oil solidifying polymer.

148. The method of clause 139 where the hydrophobic oil sorbing polymers include but are not limited to those selected from the group consisting of: block copolymers, diblock copolymers; triblock copolymers; radial block polymers, multi-block polymers, cross-linked polymers, crosslinked copolymers of hydroxypropyl cellulose acrylate/octadecyl acrylate; cross-linked polyolefin terpolymers; cross-linked octadecylacrylate-co-acrylic acid copolymers; polynorbornene polymers; styrene-ethylene/butylene-sytrene block copolymers; styrene/butadiene block copolymers; polytertiarybutylstyrene; polyacrylo-nitrile butadiene; polyisoprene (rubber), polyethylene; polypropylene; poly isobutylene; polyethylene terephthalate fibers modified by the adherence of crosslinked polymer coatings and materials and polymers sold under the trade name Organolock® N65; Norosorex®; Enviro-Bond™ 403; Enviro-Bond™ 405; Elastol 1; Elastol 2; Nochars A 610; Nochars A 650; Omni-Zorb #2000; Omni-Zorb # AZ1N; Omni-Zorb # BZ; Omni-Zorb # PZ; Petro-Lock; Clear-Tec Rubberizer®; SPI Oil Bond, Waste-set PS #3200, and Waste-set PS #3400.

149. The method of clause 139 where the man-made porous solid particles are selected from the group of thermoplastic plastic pellets including but not limited to those made with acrylonitrile butadiene styrene (ABS); polyoxymethylene (POM); low density polyethylene (LDPE); high density polyethylene (HDPE); nylon; polyether ether ketone (PEEK); polycarbonate (PC); polyethylene (PE); polypropylene (PP); polystyrene (PS); polyvinyl chloride (PVC).

150. The method of clause 139 where the porous solid particles are absorption or stripping or biofilm support type mass transfer packing materials are porous plastic, metal or ceramic and are commonly and commercially available, for example but not limited to Flexiring™, Intalox™ Snowflake, Cascade Mini-Rings™, BetaRings™ manufactured by Koch-Glitsch LP, Jaeger tri-Paks® manufacture red by Rashig USA, Inc., Coralife® Bio-Balls, or Marineland Bio-Filter Balls.

151. The method of clause 139 where the porous solid particles are structured or random porous materials with a nominal size from about 0.25 inch (6.35 mm) to about 1.5 inch (38.1 mm).

152. The method of clause 139 where the porous solid 0.25 inch (6.35 mm) to about 1.5 inch (38.1 mm) particles coated or embedded with the essential oil, fragrance, perfume, insecticide, insect repelling essential oil/granular polymer matrix are packed into a filter support housing or container that allows air to be pumped through the filter with an air blower, an enclosed ring air blower or an air pump, to volatilize the volatile organic compounds from the essential oil, fragrance, perfume, insecticide, insect repelling or essential oil extract volatile organic compound-granular polymer matrix into the air inside the filter housing and then to pump the vapor phase volatile organic compounds into an enclosed space, a semi-enclosed space or an open space.

153. The method of clause 139 where the optional gas impermeable material is selected from the group consisting of ethylene vinyl alcohol (EVA) polyester laminate; a low-density polyethylene (LDPE) polyester laminate EVA-LDPE polyester laminate; polytetrafluoroethylene (PTFE); high density polyethylene (HDPE); nylon; polyether ether ketone (PEEK); polycarbonate (PC); polyethylene (PE); polypropylene (PP); polystyrene (PS); polyvinyl chloride (PVC); and materials sold under the trade name Evolution®; Compel®; Barrier 450™; and Optima™.

154. The method of clause 139 where the ratio of essential oil, fragrance, perfume, insecticide, insect repelling or essential oil extract volatile organic carbon to granular polymer sorbent is from about 1 to about 15 on a weight basis.

155. The method of clause 139 where the ratio of the essential oil, fragrance, perfume, insecticide, insect repelling or essential oil extract volatile organic carbon plus granular polymer to solid pellet ratio is from about 0.05 to about 3.00 on a weight basis.

156. The method of clause 139 where the bulk density of the essential oil, fragrance, perfume, insecticide, insect repelling or essential oil extract volatile organic carbon/granular polymer/solid particle matrix contained within into the porous housing is from about 0.20 g/cm³ to about 2.00 g/cm³.

157. The method of clause 139 where the porous solid particles have a packed total porosity from about 0.50 to about 0.97.

158. The method of clause 139 where the essential oil, fragrance, perfume, insecticide, insect repelling or essential oil extract volatile organic carbon/granular polymer/solid particle matrix have an initial packed total porosity from about 0.50 to about 0.90.

159. The method of clause 139 where the ambient temperature of manufacture is from about 5° C. to about 45° C.

160. A method to provide aromatherapy or fragrance by the controlled sustained time-release of aromatherapy or fragrance emitting volatile organic compounds into the air by the active pumping of air through an air filter emitting volatile organic compounds into the air comprising:
a) an essential oil, fragrance, perfume or essential oil extract volatile organic compound-granular polymer matrix consisting of one or more volatile organic compounds sorbed with one or more solid polymers;
b) naturally occurring or man-made porous solid particles coated with the volatile organic compound-granular polymer matrix;
c) a gas permeable material on at least two sides of a filter housing that contains the solid particles coated with the volatile organic compound-granular polymer-solid particle mixture; and optionally;
d) a gas impermeable material on one or more sides of the filter housing that contains the solid particles coated with the volatile organic compound-granular polymer matrix.

161. The method of clause 160 wherein the essential oil, fragrance, perfume or essential oil extract volatile organic compound that has a vapor pressure from about 0.0002 mm Hg to about 40.000 mm Hg when measured at a temperature of 20° C. and a pressure of 760 mm Hg.

162. The method of clause 160 where the volatile organic compound is an essential oil.

163. The method of clause 160 wherein the essential oil, fragrance, perfume, insecticide, insect repelling or essential oil extract essential oil is selected from the group consisting of: Abies siberica; Allium sativum; Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum; Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia oliveri; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum; Pinus sylvestri; Pogostemon spp.; Pogostemon cablin; Pelargonium reniforme; Pseudotsuga menziensii; Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella or Zingiber officinale.

164. The method of clause 160 wherein the essential oil is an extract from an essential oil.

165. The method of clause 160 where the essential oil extract is selected from the group consisting of: bisabolene; borneol; 3-carene; caryophyllene oxide; camphene; camphor; carvacrol; cis-carveol; cedrol; 1,8-cineole; citral; cis-verbenol; citronellal; citronellol; eugenol; farnesol; geranial; geraniol; 4-isopropylbenzenemethanol; menthol; menthone; methyl salicylate; myrcene; limonene; linalool; p-menthane-3,8-diol; neral; nerol; patchoulol; perillaldehyde; perillyl alcohol; a-pinene; b-pinene; α-terpineol; thymol; vanillin; verbenone or vetviverol.

166. The method of clause 160 where the fragrance is a fragrance oil.

167. The method of clause 160 where the fragrance is a fragrance oil extract.

168. The method of clause 160 where the polymer is a hydrophobic oil sorbing granular polymer or a hydrophobic oil solidifying polymer.

169. The method of clause 168 where the hydrophobic oil sorbing polymers include but are not limited to those selected from the group consisting of: block copolymers, diblock copolymers; triblock copolymers; radial block polymers, multi-block polymers, cross-linked polymers, crosslinked copolymers of hydroxypropyl cellulose acrylate/octadecyl acrylate; cross-linked polyolefin terpolymers; cross-linked octadecylacrylate-co-acrylic acid copolymers; polynorbornene polymers; styrene-ethylene/butylene-sytrene block copolymers; styrene/butadiene block copolymers; polytertiarybutylstyrene; polyacrylo-nitrile butadiene; polyisoprene (rubber), polyethylene; polypropylene; poly isobutylene; polyethylene terephthalate fibers modified by the adherence of crosslinked polymer coatings and materials and polymers sold under the trade name Organolock® N65; Norosorex®; Enviro-Bond™ 403; Enviro-Bond™ 405; Elastol 1; Elastol 2; Nochars A 610; Nochars A 650; Omni-Zorb #2000; Omni-Zorb # AZ1N; Omni-Zorb # BZ; Omni-Zorb # PZ; Petro-Lock; Clear-Tec Rubberizer®; SPI Oil Bond, Waste-set PS #3200, and Waste-set PS #3400.

170. The method of clause 160 where the man-made porous solid particles are selected from the group of thermoplastic plastic pellets including but not limited to those made with acrylonitrile butadiene styrene (ABS); polyoxymethylene (POM); low density polyethylene (LDPE); high density polyethylene (HDPE); nylon; polyether ether ketone (PEEK); polycarbonate (PC); polyethylene (PE); polypropylene (PP); polystyrene (PS); polyvinyl chloride (PVC).

171. The method of clause 160 where the porous solid particles are absorption or stripping or biofilm support type mass transfer packing materials are porous plastic, metal or ceramic and are commonly and commercially available, for example but not limited to Flexiring™, Intalox™ Snowflake, Cascade Mini-Rings™, BetaRings™ manufactured by Koch-Glitsch LP, Jaeger tri-Paks® manufacture red by Rashig USA, Inc., Coralife® Bio-Balls, or Marineland Bio-Filter Balls.

172. The method of clause 160 where the porous solid particles are structured or random porous materials with a nominal size from about 0.25 inch (6.35 mm) to about 1.5 inch (38.1 mm).

173. The method of clause 160 where the porous solid 0.25 inch (6.35 mm) to about 1.5 inch (38.1 mm) particles coated or embedded with the essential oil, fragrance, perfume, insecticide, insect repelling essential oil/granular polymer matrix are packed into a filter support housing or container that allows air to be pumped through the filter with an air blower, an enclosed ring air blower or an air pump, to volatilize the volatile organic compounds from the essential oil, fragrance, perfume, insecticide, insect repelling or essential oil extract volatile organic compound-granular polymer matrix into the air inside the filter housing and then to pump the vapor phase volatile organic compounds into an enclosed space, a semi-enclosed space or an open space.

174. The method of clause 160 where the optional gas impermeable material is selected from the group consisting of ethylene vinyl alcohol (EVA) polyester laminate; a low-density polyethylene (LDPE) polyester laminate EVA-LDPE polyester laminate; polytetrafluoroethylene (PTFE); high density polyethylene (HDPE); nylon; polyether ether ketone (PEEK); polycarbonate (PC); polyethylene (PE); polypropylene (PP); polystyrene (PS); polyvinyl chloride (PVC); and materials sold under the trade name Evolution®; Compel®; Barrier 450™; and Optima™.

175. The method of clause 160 where the ratio of essential oil, fragrance, perfume or essential oil extract volatile organic carbon to granular polymer sorbent is from about 1 to about 15 on a weight basis.

176. The method of clause 160 where the ratio of the essential oil, fragrance, perfume or essential oil extract volatile organic carbon plus granular polymer to solid pellet ratio is from about 0.05 to about 3.00 on a weight basis.

177. The method of clause 160 where the bulk density of the essential oil, fragrance, perfume or essential oil extract volatile organic carbon/granular polymer/solid particle matrix contained within into the porous housing is from about 0.20 g/cm³ to about 2.00 g/cm³.

178. The method of clause 160 where the porous solid particles have a packed total porosity from about 0.50 to about 0.97.

179. The method of clause 160 where the essential oil, fragrance, perfume or essential oil extract volatile organic carbon/polymer/solid particle matrix have an initial packed total porosity from about 0.20 to about 0.90.

180. The method of clause 160 where the ambient temperature of manufacture is from about 5° C. to about 45° C.

181. A device that is manufactured at ambient temperatures used to repels or inactivate insects or other arthropods by the passive, slow, sustained time-release of repelling, insecticidal or fragrance emitting volatile organic compounds into the air comprising:
a) an essential oil, fragrance, perfume, insecticide, insect repelling or essential oil extract volatile organic compound-granular polymer-activated carbon matrix consisting of one or more volatile organic compounds sorbed with one or more granular polymers and one or more activated carbons;
b) naturally occurring or man-made porous or non-porous solid particles coated with the volatile organic compound-granular polymer-powdered activated carbon matrix;
c) a gas permeable, gas semi-permeable or a microporous material on at least one side of the structure that contains the solid particles coated with the volatile organic compound-granular polymer-activated carbon-solid particle mixture; and optionally
d) a gas impermeable material on one or more sides of the material that contains the solid particles coated with the volatile organic compound-granular polymer-activated carbon matrix.

182. The device of clause 181 wherein the essential oil, fragrance, perfume, insecticide, insect repelling or essential oil extract volatile organic compound that repels or inactivates insects or other arthropods has a vapor pressure from about 0.0002 mm Hg to about 40.000 mm Hg when measured at a temperature of 20° C. and a pressure of 760 mm Hg.

183. The device of clause 181 where the volatile organic compound is an essential oil.

184. The device of clause 181 wherein the essential oil, fragrance, perfume, insecticide, insect repelling or essential oil extract essential oil is selected from the group consisting of: Abies siberica; Allium sativum; Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum; Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia oliveri; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum; Pinus sylvestri; Pogostemon spp.; Pogostemon cablin; Pelargonium reniforme; Pseudotsuga menziensii; Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella or Zingiber officinale.

185. The device of clause 183 wherein the essential oil is an extract from an essential oil.

186. The device of clause 185 where the essential oil extract is selected from the group consisting of: bisabolene; borneol; 3-carene; caryophyllene oxide; camphene; camphor; carvacrol; cis-carveol; cedrol; 1,8-cineole; citral; cis-verbenol; citronellal; citronellol; eugenol; farnesol; geranial; geraniol; 4-isopropylbenzenemethanol; menthol; menthone; methyl salicylate; myrcene; limonene; linalool; p-menthane-3,8-diol; neral; nerol; patchoulol; perillaldehyde; perillyl alcohol; a-pinene; b-pinene; α-terpineol; thymol; vanillin; verbenone or vetviverol.

187. The device of clause 181 where the insecticidal volatile organic compound is an insecticide.

188. The device of clause 187 where the insecticidal volatile organic compound is selected from the group consisting of: dichlorvos; DEET (N,N-diethyl toluamide); etofenprox; or ethyl hexanediol.

189. The device of clause 181 where the granular polymer is a hydrophobic oil sorbing polymer or a hydrophobic oil solidifying polymer.

190. The device of clause 189 where the hydrophobic oil sorbing polymers include but are not limited to those selected from the group consisting of: block copolymers, diblock copolymers; triblock copolymers; radial block polymers, multi-block polymers, cross-linked polymers, crosslinked copolymers of hydroxypropyl cellulose acrylate/octadecyl acrylate; cross-linked polyolefin terpolymers; cross-linked octadecylacrylate-co-acrylic acid copolymers; polynorbornene polymers; styrene-ethylene/butylene-sytrene block copolymers; styrene/butadiene block copolymers; polytertiarybutylstyrene; polyacrylo-nitrile butadiene; polyisoprene (rubber), polyethylene; polypropylene; poly isobutylene; polyethylene terephthalate fibers modified by the adherence of crosslinked polymer coatings and materials and polymers sold under the trade name Organolock® N65; Norosorex®; Enviro-Bond™ 403; Enviro-Bond™ 405; Elastol 1; Elastol 2; Nochars A 610; Nochars A 650; Omni-Zorb #2000; Omni-Zorb # AZ1N; Omni-Zorb # BZ; Omni-Zorb # PZ; Petro-Lock; Clear-Tec Rubberizer®; SPI Oil Bond, Waste-set PS #3200, and Waste-set PS #3400.

191. The device of clause 181 where the activated carbon is a powdered activated carbon.

192. The device of clause 181 where the activated carbon is a granular activated carbon.

193. The device of clause 181 where the man-made porous or non-porous solid particles are selected from the group of thermoplastic plastic pellets including but not limited to those made with acrylonitrile butadiene styrene (ABS); polyoxymethylene (POM); low density polyethylene (LDPE); high density polyethylene (HDPE); nylon; polyether ether ketone (PEEK); polycarbonate (PC); polyethylene (PE); polypropylene (PP); polystyrene (PS); polyvinyl chloride (PVC).

194. The device of clause 181 where the solid particles are made from natural materials selected from the group of natural wood pellets; pea-stone gravel; biomass pellets; coffee husk pellets; hemp pellets; corn cob pellets.

195. The device of clause 181 where the solid pellets are either a porous or non-porous with particle sizes varying in length, width and depth dimensions from for example from 1.0 mm to 5.0 mm.

196. The device of clause 181 where the gas permeable, gas semi-permeable or microporous with materials are selected from the group consisting of gas permeable woven fabrics, non-woven fabrics, injected molded gas permeable plastic mesh materials, gas permeable housings, selected from the group consisting of natural and synthetic fabrics consisting of: wool; cotton; bamboo; hemp; nylon; polyester nylon; polyester; acrylic; rayon; polyolefin; and materials sold under the trade name Gore-Tex®; Tyvek®; Evolution®; or Optima™.

197. The device of clause 181 where the optional gas impermeable material is selected from the group consisting of ethylene vinyl alcohol (EVA) polyester laminate; a low-density polyethylene (LDPE) polyester laminate EVA-LDPE polyester laminate; polytetrafluoroethylene (PTFE); high density polyethylene (HDPE); nylon; polyether ether ketone (PEEK); polycarbonate (PC); polyethylene (PE); polypropylene (PP); polystyrene (PS); polyvinyl chloride (PVC); and materials sold under the trade name Evolution®; Compel®; Barrier 450™; and Optima™.

198. The device of clause 181 where the ratio of essential oil, fragrance, perfume, insecticide, insect repelling or essential oil extract volatile organic carbon to granular polymer sorbent-activated carbon mixture is from about 1 to about 15 on a weight basis.

199. The device of clause 181 where the ratio of granular polymer to activated carbon is from about 1 to 1 to about 10 to 1.

200. The device of clause 181 where the ratio of the essential oil, fragrance, perfume, insecticide, insect repelling or essential oil extract volatile organic carbon plus granular polymer and activated carbon to solid pellet ratio is from about 0.05 to about 3.00 on a weight basis.

201. The device of clause 181 where the bulk density of the essential oil, fragrance, perfume, insecticide, insect repelling or essential oil extract volatile organic carbon/granular polymer/activated carbon/solid particle matrix contained within into the porous housing is from about 0.20 g/cm³ to about 2.00 g/cm³.

202. The device of clause 181 where the porous solid particles have a packed total porosity from about 0.50 to about 0.97.

203. The device of clause 181 where the essential oil, fragrance, perfume, insecticide, insect repelling or essential oil extract volatile organic carbon/granular polymer/activated carbon/solid particle matrix have an initial packed total porosity from about 0.20 to about 0.90.

204. The device of clause 181 where the ambient temperature of manufacture is from about 5° C. to about 45° C.

205. A method to repel or inactivate insects or other arthropods by the active pumping of air through an air filter for the controlled, sustained time-release of repelling, insecticidal or fragrance emitting volatile organic compounds into the air comprising:
a) an essential oil, fragrance, perfume, insecticide, insect repelling or essential oil extract volatile organic compound-granular polymer matrix consisting of one or more volatile organic compounds sorbed with one or more solid polymers and one or more activated carbons;
b) naturally occurring or man-made porous or non-porous solid particles coated with the volatile organic compound-granular polymer-activated carbon matrix;
c) a gas permeable material on at least two sides of a filter housing that contains the solid particles coated with the volatile organic compound-polymer-activated carbon-solid particle mixture; and optionally
d) a gas impermeable material on one or more sides of the filter housing that contains the solid particles coated with the volatile organic compound-granular polymer-activated carbon matrix.

206. The method of clause 205 wherein the essential oil, fragrance, perfume, insecticide, insect repelling or essential oil extract volatile organic compound that repels or inactivates insects or other arthropods has a vapor pressure from about 0.0002 mm Hg to about 40.000 mm Hg when measured at a temperature of 20° C. and a pressure of 760 mm Hg.

207. The method of clause 205 where the volatile organic compound is an essential oil.

208. The method of clause 207 wherein the essential oil, fragrance, perfume, insecticide, insect repelling or essential oil extract essential oil is selected from the group consisting of: Abies siberica; Allium sativum; Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum; Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia oliveri; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum; Pinus sylvestri; Pogostemon spp.; Pogostemon cablin; Pelargonium reniforme; Pseudotsuga menziensii; Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella or Zingiber officinale.

209. The method of clause 205 wherein the essential oil is an extract from an essential oil.

210. The method of clause 209 where the essential oil extract is selected from the group consisting of: bisabolene; borneol; 3-carene; caryophyllene oxide; camphene; camphor; carvacrol; cis-carveol; cedrol; 1,8-cineole; citral; cis-verbenol; citronellal; citronellol; eugenol; farnesol; geranial; geraniol; 4-isopropylbenzenemethanol; menthol; menthone; methyl salicylate; myrcene; limonene; linalool; p-menthane-3,8-diol; neral; nerol; patchoulol; perillaldehyde; perillyl alcohol; a-pinene; b-pinene; α-terpineol; thymol; vanillin; verbenone or vetviverol.

211. The method of clause 205 where the insecticidal volatile organic compound is an insecticide.

212. The method of clause 211 where the insecticidal volatile organic compound is selected from the group consisting of: dichlorvos; DEET (N,N-diethyl toluamide); etofenprox; or ethyl hexanediol.

213. The method of clause 205 where the polymer is a hydrophobic oil sorbing polymer or a hydrophobic oil solidifying polymer.

214. The method of clause 213 where the hydrophobic oil sorbing polymers include but are not limited to those selected from the group consisting of: block copolymers, diblock copolymers; triblock copolymers; radial block polymers, multi-block polymers, cross-linked polymers, crosslinked copolymers of hydroxypropyl cellulose acrylate/octadecyl acrylate; cross-linked polyolefin terpolymers; cross-linked octadecylacrylate-co-acrylic acid copolymers; polynorbornene polymers; styrene-ethylene/butylene-sytrene block copolymers; styrene/butadiene block copolymers; polytertiarybutylstyrene; polyacrylo-nitrile butadiene; polyisoprene (rubber), polyethylene; polypropylene; poly isobutylene; polyethylene terephthalate fibers modified by the adherence of crosslinked polymer coatings and materials and polymers sold under the trade name Organolock® N65; Norosorex®; Enviro-Bond™ 403; Enviro-Bond™ 405; Elastol 1; Elastol 2; Nochars A 610; Nochars A 650; Omni-Zorb #2000; Omni-Zorb # AZ1N; Omni-Zorb # BZ; Omni-Zorb # PZ; Petro-Lock; Clear-Tec Rubberizer®; SPI Oil Bond, Waste-set PS #3200, and Waste-set PS #3400.

215. The method of clause 205 where the activated carbon is a powdered activated carbon.

216. The method of clause 205 where the activated carbon is a granular activated carbon.

217. The method of clause 205 where the man-made porous or non-porous solid particles are selected from the group of thermoplastic plastic pellets including but not limited to those made with acrylonitrile butadiene styrene (ABS); polyoxymethylene (POM); low density polyethylene (LDPE); high density polyethylene (HDPE); nylon; polyether ether ketone (PEEK); polycarbonate (PC); polyethylene (PE); polypropylene (PP); polystyrene (PS); polyvinyl chloride (PVC).

218. The method of clause 205 where the porous or non-porous solid particles are absorption, stripping or biofilm support type mass transfer packing materials are porous plastic, metal or ceramic and are commonly and commercially available, for example but not limited to Flexiring™, Intalox™ Snowflake, Cascade Mini-Rings™, BetaRings™ manufactured by Koch-Glitsch LP, Jaeger tri-Paks® manufactured by Rashig USA, Inc., Coralife® Bio-Balls, or Marineland Bio-Filter Balls.

219. The method of clause 205 where the porous or non-porous solid particles are structured or random porous materials with a nominal size from about 0.25 inch (6.35 mm) to about 1.5 inch (38.1 mm).

220. The method of clause 205 where the porous or non-porous solid 0.25 inch (6.35 mm) to about 1.5 inch (38.1 mm) particles coated or embedded with the essential oil, fragrance, perfume, insecticide, insect repelling essential oil/granular polymer-activated carbon matrix are packed into a filter support housing or container that allows air to be pumped through the filter with an air blower, an enclosed ring air blower or an air pump, to volatilize the volatile organic compounds from the essential oil, fragrance, perfume, insecticide, insect repelling or essential oil extract volatile organic compound/granular polymer/activated carbon matrix into the air inside the filter housing and then to pump the vapor phase volatile organic compounds into an enclosed space, a semi-enclosed space or an open space.

221. The method of clause 205 where the optional gas impermeable material is selected from the group consisting of ethylene vinyl alcohol (EVA) polyester laminate; a low-density polyethylene (LDPE) polyester laminate EVA-LDPE polyester laminate; polytetrafluoroethylene (PTFE); high density polyethylene (HDPE); nylon; polyether ether ketone (PEEK); polycarbonate (PC); polyethylene (PE); polypropylene (PP); polystyrene (PS); polyvinyl chloride (PVC); and materials sold under the trade name Evolution®; Compel®; Barrier 450™; and Optima™.

222. The method of clause 205 where the ratio of essential oil, fragrance, perfume, insecticide, insect repelling or essential oil extract volatile organic carbon to the granular polymer-activated carbon sorbent mixture is from about 1 to about 15 on a weight basis.

223. The method of clause 205 where the ratio of the essential oil, fragrance, perfume, insecticide, insect repelling or essential oil extract volatile organic carbon plus granular polymer-activated carbon mixture to solid particle ratio is from about 0.05 to about 3.00 on a weight basis.

224. The method of clause 205 where the bulk density of the essential oil, fragrance, perfume, insecticide, insect repelling or essential oil extract volatile organic carbon/granular polymer/activated carbon solid particle matrix contained within into the porous housing is from about 0.20 g/cm³ to about 2.0 g/cm³.

225. The method of clause 205 where the porous or non-porous solid particles have a packed total porosity from about 0.50 to about 0.97.

226. The method of clause 205 where the essential oil, fragrance, perfume, insecticide, insect repelling or essential oil extract volatile organic carbon/granular polymer/activated carbon porous or non-porous particle matrix has an initial packed total porosity from about 0.20 to about 0.90.

227. The method of clause 205 where the ambient temperature of manufacture is from about 5° C. to about 45° C.

228. A device that is manufactured at ambient temperatures used to repels or inactivate insects or other arthropods by the passive, slow, sustained time-release of repelling, insecticidal or fragrance emitting volatile organic compounds into the air comprising:
a) an essential oil, fragrance, perfume, insecticide, insect repelling or essential oil extract volatile organic compound-granular polymer-activated carbon matrix consisting of one or more volatile organic compounds sorbed with one or more granular polymers and one or more activated carbons;
b) a gas permeable, gas semi-permeable or a microporous material on at least one side of the structure that contains volatile organic compound-granular polymer-activated carbon matrix; and optionally;
c) a gas impermeable material on one or more sides of the material that volatile organic compound-granular polymer-activated carbon matrix.

229. The device of clause 228 wherein the essential oil, fragrance, perfume, insecticide, insect repelling or essential oil extract volatile organic compounds that repels or inactivates insects or other arthropods have vapor pressures from about 0.0002 mm Hg to about 40.000 mm Hg when measured at a temperature of 20° C. and a pressure of 760 mm Hg.

230. The device of clause 228 where the volatile organic compound is an essential oil.

231. The device of clause 230 wherein the essential oil, fragrance, perfume, insecticide, insect repelling or essential oil extract essential oil is selected from the group consisting of: Abies siberica; Allium sativum; Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum; Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia oliveri; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum; Pinus sylvestri; Pogostemon spp.; Pogostemon cablin; Pelargonium reniforme; Pseudotsuga menziensii; Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella or Zingiber officinale.

232. The device of clause 228 wherein the essential oil is an extract from an essential oil.

233. The device of clause 232 where the essential oil extract is selected from the group consisting of: borneol; 3-carene; caryophyllene oxide; carvacrol; cis-carveol; 1,8-cineole; cis-verbenol; citronellal; eugenol; geraniol; 4-isopropylbenzenemethanol; menthol; methyl salicylate; myrcene; limonene; linalool; p-menthane-3,8-diol; perillaldehyde; perillyl alcohol; a-pinene; b-pinene; α-terpineol; or thymol.

234. The device of clause 228 where the granular polymer is a hydrophobic oil sorbing polymer or a hydrophobic oil solidifying polymer.

235. The device of clause 234 where the hydrophobic oil sorbing polymers include but are not limited to those selected from the group consisting of: block copolymers, diblock copolymers; triblock copolymers; radial block polymers, multi-block polymers, cross-linked polymers, crosslinked copolymers of hydroxypropyl cellulose acrylate/octadecyl acrylate; cross-linked polyolefin terpolymers; cross-linked octadecylacrylate-co-acrylic acid copolymers; polynorbornene polymers; styrene-ethylene/butylene-sytrene block copolymers; styrene/butadiene block copolymers; polytertiarybutylstyrene; polyacrylo-nitrile butadiene; polyisoprene (rubber), polyethylene; polypropylene; poly isobutylene; polyethylene terephthalate fibers modified by the adherence of crosslinked polymer coatings and materials and polymers sold under the trade name Organolock® N65; Norosorex®; Enviro-Bond™ 403; Enviro-Bond™ 405; Elastol 1; Elastol 2; Nochars A 610; Nochars A 650; Omni-Zorb #2000; Omni-Zorb # AZ1N; Omni-Zorb # BZ; Omni-Zorb # PZ; Petro-Lock; Clear-Tec Rubberizer®; SPI Oil Bond, Waste-set PS #3200, and Waste-set PS #3400.

236. The device of clause 228 where the activated carbon is a powdered activated carbon.

237. The device of clause 228 where the activated carbon is a granular activated carbon.

238. The device of clause 228 where the gas permeable, gas semi-permeable or microporous with materials are selected from the group consisting of gas permeable woven fabrics, non-woven fabrics, injected molded gas permeable plastic mesh materials, gas permeable housings, selected from the group consisting of natural and synthetic fabrics consisting of: wool; cotton; bamboo; hemp; nylon; polyester nylon; polyester; acrylic; rayon; polyolefin; and materials sold under the trade name Gore-Tex®; Tyvek®; Evolution®; or Optima™.

239. The device of clause 228 where the optional gas impermeable material is selected from the group consisting of ethylene vinyl alcohol (EVA) polyester laminate; a low-density polyethylene (LDPE) polyester laminate EVA-LDPE polyester laminate; polytetrafluoroethylene (PTFE); high density polyethylene (HDPE); nylon; polyether ether ketone (PEEK); polycarbonate (PC); polyethylene (PE); polypropylene (PP); polystyrene (PS); polyvinyl chloride (PVC); and materials sold under the trade name Evolution®; Compel®; Barrier 450™; and Optima™.

240. The device of clause 228 where the ratio of essential oil, fragrance, perfume, insecticide, insect repelling or essential oil extract volatile organic carbon to granular polymer sorbent-activated carbon mixture is from about 1 to about 15 on a weight basis.

241. The device of clause 228 where the bulk density of the essential oil, fragrance, perfume, insecticide, insect repelling or essential oil extract volatile organic carbon/granular polymer/activated carbon matrix contained within in the porous housing is from about 0.50 g/cm³ to about 1.8 g/cm³.

242. The device of clause 228 here the ambient temperature of manufacture is from about 5° C. to about 45° C.

243. A device that is manufactured at ambient temperatures used for aromatherapy or introducing a fragrance by the passive, slow, sustained time-release emissions of aromatherapy or fragrance volatile organic compounds into the air comprising:
a) an aromatherapy or fragrance volatile organic compound-granular polymer-solid particle matrix consisting of one or more aromatherapy or fragrance volatile organic compounds sorbed with one or more solid polymers;
b) naturally occurring or man-made porous or non-porous solid particles coated with the volatile organic compound-granular polymer matrix;
c) a structure that contains aromatherapy or fragrance volatile organic compound-granular polymer coated solid particle matrix that can be opened to the atmosphere on one or more sides; and
d) a structure that contains the aromatherapy or fragrance volatile organic compound-granular polymer coated solid particle matrix that consists of a gas impermeable sealed or closed to the atmosphere on one or more sides.

244. The device of clause 243 wherein the aromatherapy or fragrance volatile organic compound that provides aromatherapy or fragrance has a vapor pressure from about 0.0002 mm Hg to about 40.000 mm Hg when measured at a temperature of 20° C. and a pressure of 760 mm Hg.

245. The device of clause 243 where the volatile organic compound is an essential oil.

246. The device of clause 245 wherein the essential oil, fragrance, perfume, insecticide, insect repelling or essential oil extract essential oil is selected from the group consisting of: Abies siberica; Allium sativum; Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum; Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia oliveri; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum; Pinus sylvestri; Pogostemon spp.; Pogostemon cablin; Pelargonium reniforme; Pseudotsuga menziensii; Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella or Zingiber officinale.

247. The device of clause 243 wherein the essential oil is an extract from an essential oil.

248. The device of clause 247 where the essential oil extract is selected from the group consisting of: bisabolene; borneol; 3-carene; caryophyllene oxide; camphene; camphor; carvacrol; cis-carveol; cedrol; 1,8-cineole; citral; cis-verbenol; citronellal; citronellol; eugenol; farnesol; geranial; geraniol; 4-isopropylbenzenemethanol; menthol; menthone; methyl salicylate; myrcene; limonene; linalool; p-menthane-3,8-diol; neral; nerol; patchoulol; perillaldehyde; perillyl alcohol; a-pinene; b-pinene; α-terpineol; thymol; vanillin; verbenone or vetviverol.

249. The device of clause 243 is a fragrance oil.

250. The device of clause 243 where the granular polymer is a hydrophobic oil sorbing polymer or a hydrophobic oil solidifying polymer.

251. The device of clause 250 where the hydrophobic oil sorbing polymers include but are not limited to those selected from the group consisting of: block copolymers, diblock copolymers; triblock copolymers; radial block polymers, multi-block polymers, cross-linked polymers, crosslinked copolymers of hydroxypropyl cellulose acrylate/octadecyl acrylate; cross-linked polyolefin terpolymers; cross-linked octadecylacrylate-co-acrylic acid copolymers; polynorbornene polymers; styrene-ethylene/butylene-sytrene block copolymers; styrene/butadiene block copolymers; polytertiarybutylstyrene; polyacrylo-nitrile butadiene; polyisoprene (rubber), polyethylene; polypropylene; poly isobutylene; polyethylene terephthalate fibers modified by the adherence of crosslinked polymer coatings and materials and polymers sold under the trade name Organolock® N65; Norosorex®; Enviro-Bond™ 403; Enviro-Bond™ 405; Elastol 1; Elastol 2; Nochars A 610; Nochars A 650; Omni-Zorb #2000; Omni-Zorb # AZ1N; Omni-Zorb # BZ; Omni-Zorb # PZ; Petro-Lock; Clear-Tec Rubberizer®; SPI Oil Bond, Waste-set PS #3200, and Waste-set PS #3400.

252. The device of clause 243 where the man-made porous or non-porous solid particles are selected from the group of thermoplastic plastic pellets including but not limited to those made with acrylonitrile butadiene styrene (ABS); polyoxymethylene (POM); low density polyethylene (LDPE); high density polyethylene (HDPE); nylon; polyether ether ketone (PEEK); polycarbonate (PC); polyethylene (PE); polypropylene (PP); polystyrene (PS); polyvinyl chloride (PVC).

253. The device of clause 243 where the solid particles are made from natural materials selected from the group of natural wood pellets; pea-stone gravel; biomass pellets; coffee husk pellets; hemp pellets; corn cob pellets.

254. The device of clause 243 where the solid pellets are either a porous or non-porous with particle sizes varying in length, width and depth dimensions from for example from 1.0 mm to 5.0 mm.

255. The device of clause 243 where the optional gas impermeable material is selected from the group consisting of glass, ethylene vinyl alcohol (EVA) polyester laminate; a low-density polyethylene (LDPE) polyester laminate EVA-LDPE polyester laminate; polyethylene: terephthalate: (PETE); Polytetrafluoroethylene (PTFE); high density polyethylene (HDPE); nylon; polyether ether ketone (PEEK); polycarbonate (PC); polyethylene (PE); polypropylene (PP); polystyrene (PS); polyvinyl chloride (PVC); and materials sold under the trade name Evolution®; Compel®; Barrier 450™; and Optima™.

256. The device of clause 243 where the ratio of aromatherapy essential oil, fragrance, perfume, or essential oil extract volatile organic carbon to granular polymer sorbent mixture is from about 1 to about 15 on a weight basis.

257. The device of clause 243 where the bulk density of the of aromatherapy essential oil, fragrance, perfume, or essential oil extract volatile organic carbon/granular polymer matrix/solid particle matrix contained within structure is from about 0.50 g/cm³ to about 1.8 g/cm³.

258. The device of clause 243 where the ambient temperature of manufacture is from about 5° C. to about 45° C.

259. A device that is manufactured at ambient temperatures used for aromatherapy or introducing a fragrance by the passive, slow, sustained time-release emissions of aromatherapy or fragrance volatile organic compounds into the air comprising:
a) an aromatherapy or fragrance volatile organic compound-granular polymer matrix consisting of one or more aromatherapy or fragrance volatile organic compounds sorbed with one or more solid polymers;
b) a structure that contains the aromatherapy or fragrance volatile organic compound-granular polymer matrix that can be opened to the atmosphere on one or more sides; and
c) a structure that contains the aromatherapy or fragrance volatile organic compound-granular polymer matrix that consists of a gas impermeable material sealed or closed to the atmosphere on one or more sides.

260. The device of clause 259 wherein the aromatherapy or fragrance volatile organic compound that provides aromatherapy or fragrance has a vapor pressure from about 0.0002 mm Hg to about 40.000 mm Hg when measured at a temperature of 20° C. and a pressure of 760 mm Hg.

261. The device of clause 259 where the volatile organic compound is an essential oil.

262. The device of clause 261 wherein the essential oil, fragrance, perfume, insecticide, insect repelling or essential oil extract essential oil is selected from the group consisting of: Abies siberica; Allium sativum; Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum; Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia oliveri; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum; Pinus sylvestri; Pogostemon spp.; Pogostemon cablin; Pelargonium reniforme; Pseudotsuga menziensii; Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella or Zingiber officinale.

263. The device of clause 259 wherein the essential oil is an extract from an essential oil.

264. The device of clause 263 where the essential oil extract is selected from the group consisting of: bisabolene; borneol; 3-carene; caryophyllene oxide; camphene; camphor; carvacrol; cis-carveol; cedrol; 1,8-cineole; citral; cis-verbenol; citronellal; citronellol; eugenol; farnesol; geranial; geraniol; 4-isopropylbenzenemethanol; menthol; menthone; methyl salicylate; myrcene; limonene; linalool; p-menthane-3,8-diol; neral; nerol; patchoulol; perillaldehyde; perillyl alcohol; a-pinene; b-pinene; α-terpineol; thymol; vanillin; verbenone or vetviverol.

265. The device of clause 259 is a fragrance oil.

266. The device of clause 259 where the granular polymer is a hydrophobic oil sorbing polymer or a hydrophobic oil solidifying polymer.

267. The device of clause 266 where the hydrophobic oil sorbing polymers include but are not limited to those selected from the group consisting of: block copolymers, diblock copolymers; triblock copolymers; radial block polymers, multi-block polymers, cross-linked polymers, crosslinked copolymers of hydroxypropyl cellulose acrylate/octadecyl acrylate; cross-linked polyolefin terpolymers; cross-linked octadecylacrylate-co-acrylic acid copolymers; polynorbornene polymers; styrene-ethylene/butylene-sytrene block copolymers; styrene/butadiene block copolymers; polytertiarybutylstyrene; polyacrylonitrile butadiene; polyisoprene (rubber), polyethylene; polypropylene; poly isobutylene; polyethylene terephthalate fibers modified by the adherence of crosslinked polymer coatings and materials and polymers sold under the trade name Organolock® N65; Norosorex®; Enviro-Bond™ 403; Enviro-Bond™ 405; Elastol 1; Elastol 2; Nochars A 610; Nochars A 650; Omni-Zorb #2000; Omni-Zorb # AZ1N; Omni-Zorb # BZ; Omni-Zorb # PZ; Petro-Lock; Clear-Tec Rubberizer®; SPI Oil Bond, Waste-set PS #3200, and Waste-set PS #3400.

268. The device of clause 259 where the gas impermeable material is selected from the group consisting of glass, ethylene vinyl alcohol (EVA) polyester laminate; a low-density polyethylene (LDPE) polyester laminate EVA-LDPE polyester laminate; polyethylene terephthalate (PETE); Polytetrafluoroethylene (PTFE); high density polyethylene (HDPE); nylon; polyether ether ketone (PEEK); polycarbonate (PC); polyethylene (PE); polypropylene (PP); polystyrene (PS); polyvinyl chloride (PVC); and materials sold under the trade name Evolution®; Compel®; Barrier 450™; and Optima™.

269. The device of clause 259 where the ratio of aromatherapy essential oil, fragrance, perfume, or essential oil extract volatile organic carbon to granular polymer sorbent mixture is from about 1 to about 15 on a weight basis.

270. The device of clause 259 where the bulk density of the of aromatherapy essential oil, fragrance, perfume, or essential oil extra volatile organic carbon/granular polymer matrix contained within into the structure is from about 0.50 g/cm³ to about 1.8 g/cm³.

271. The device of clause 259 where the ambient temperature of manufacture is from about 5° C. to about 45° C.

272. A device that is manufactured at ambient temperatures used to repels or inactivate insects or other arthropods by the passive, slow, sustained time-release of repelling, insecticidal or fragrance emitting volatile organic compounds into the air comprising:
a) an essential oil, fragrance, perfume, insecticide, insect repelling or essential oil extract volatile organic compound-granular polymer matrix consisting of one or more volatile organic compounds sorbed with one or more granular polymers;
b) a gas permeable, gas semi-permeable or a microporous material on at least one side of the structure that contains essential oil, fragrance, perfume, insecticide, insect repelling or essential oil extract volatile organic compound-granular polymer matrix; and optionally
c) a gas impermeable material on one or more sides of the material that contains the solid particles coated with the volatile organic compound-granular polymer matrix.

273. The device of clause 272 wherein the essential oil, fragrance, perfume, insecticide, insect repelling or essential oil extract volatile organic compound that repels or inactivates insects or other arthropods has a vapor pressure from about 0.0002 mm Hg to about 40.000 mm Hg when measured at a temperature of 20° C. and a pressure of 760 mm Hg.

274. The device of clause 272 wherein the volatile organic compound is an essential oil.

275. The device of clause 274 wherein the essential oil, fragrance, perfume, insecticide, insect repelling or essential oil extract essential oil is selected from the group consisting of: Abies siberica; Allium sativum; Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum; Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia oliveri; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum; Pinus sylvestri; Pogostemon spp.; Pogostemon cablin; Pelargonium reniforme; Pseudotsuga menziensii; Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella or Zingiber officinale.

276. The device of clause 272 wherein the essential oil is an extract from an essential oil.

277. The device of clause 276 where the essential oil extract is selected from the group consisting of: bisabolene; borneol; 3-carene; caryophyllene oxide; camphene; camphor; carvacrol; cis-carveol; cedrol; 1,8-cineole; citral; cis-verbenol; citronellal; citronellol; eugenol; farnesol; geranial; geraniol; 4-isopropylbenzenemethanol; menthol; menthone; methyl salicylate; myrcene; limonene; linalool; p-menthane-3,8-diol; neral; nerol; patchoulol; perillaldehyde; perillyl alcohol; a-pinene; b-pinene; α-terpineol; thymol; vanillin; verbenone or vetviverol.

278. The device of clause 272 where the insecticidal volatile organic compound is an insecticide.

279. The device of clause 278 where the insecticidal volatile organic compound is selected from the group consisting of: dichlorvos; DEET (N,N-diethyl toluamide); etofenprox; or ethyl hexanediol.

280. The device of clause 272 where the granular polymer is a hydrophobic oil sorbing polymer or a hydrophobic oil solidifying polymer.

281. The device of clause 273 where the hydrophobic oil sorbing polymers include but are not limited to those selected from the group consisting of: block copolymers, diblock copolymers; triblock copolymers; radial block polymers, multi-block polymers, cross-linked polymers, crosslinked copolymers of hydroxypropyl cellulose acrylate/octadecyl acrylate; cross-linked polyolefin terpolymers; cross-linked octadecylacrylate-co-acrylic acid copolymers; polynorbornene polymers; styrene-ethylene/butylene-sytrene block copolymers; styrene/butadiene block copolymers; polytertiarybutylstyrene; polyacrylo-nitrile butadiene; polyisoprene (rubber), polyethylene; polypropylene; poly isobutylene; polyethylene terephthalate fibers modified by the adherence of crosslinked polymer coatings and materials and polymers sold under the trade name Organolock® N65; Norosorex®; Enviro-Bond™ 403; Enviro-Bond™ 405; Elastol 1; Elastol 2; Nochars A 610; Nochars A 650; Omni-Zorb #2000; Omni-Zorb # AZ1N; Omni-Zorb # BZ; Omni-Zorb # PZ; Petro-Lock; Clear-Tec Rubberizer®; SPI Oil Bond, Waste-set PS #3200, and Waste-set PS #3400.

282. The device of clause 272 where the gas permeable, gas semi-permeable or microporous with materials are selected from the group consisting of gas permeable woven fabrics, non-woven fabrics, injected molded gas permeable plastic mesh materials, gas permeable housings, selected from the group consisting of natural and synthetic fabrics consisting of: wool; cotton; bamboo; hemp; nylon; polyester nylon; polyester; acrylic; rayon; polyolefin; and materials sold under the trade name Gore-Tex®; Tyvek®; Evolution®; or Optima™.

283. The device of clause 272 where the optional gas impermeable material is selected from the group consisting of ethylene vinyl alcohol (EVA) polyester laminate; a low-density polyethylene (LDPE) polyester laminate EVA-LDPE polyester laminate; polytetrafluoroethylene (PTFE); high density polyethylene (HDPE); nylon; polyether ether ketone (PEEK); polycarbonate (PC); polyethylene (PE); polypropylene (PP); polystyrene (PS); polyvinyl chloride (PVC); and materials sold under the trade name Evolution®; Compel®; Barrier 450™; and Optima™.

284. The device of clause 272 where the ratio of essential oil, fragrance, perfume, insecticide, insect repelling or essential oil extract volatile organic carbon to granular polymer sorbent is from about 1 to about 15 on a weight basis.

285. The device of clause 272 where the bulk density of the essential oil, fragrance, perfume, insecticide, insect repelling or essential oil extract volatile organic carbon/granular polymer matrix contained within into the porous housing is from about 0.20 g/cm³ to about 2.0 g/cm³.

286. The device of clause 272 where the essential oil, fragrance, perfume, insecticide, insect repelling or essential oil extract volatile organic carbon/granular polymer matrix have an initial packed total porosity from about 0.01 to about 0.55.

287. The device of clause 272 where the ambient temperature of manufacture is from about 5° C. to about 45° C.

### EXAMPLES

The following examples further describe and demonstrate embodiments with the scope of the present disclosure. The examples are given solely for the purpose of illustration and are not be constructed as limitations of the present disclosure as many variations are possible without departing from the spirit and scope of the disclosure.

### Example 1

### Insect Repellent Device

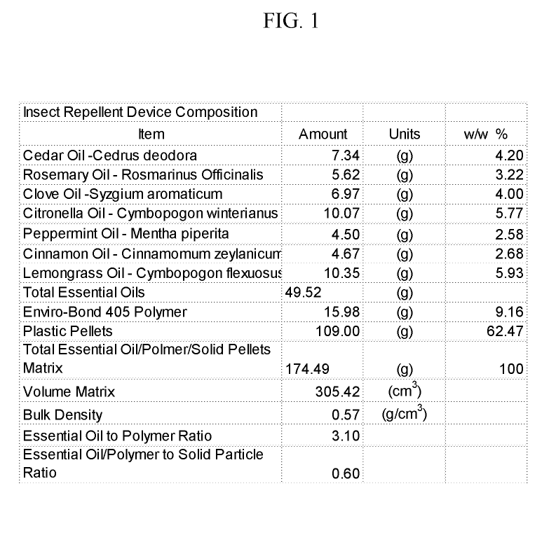

In a suitable sized vessel combine plastic polypropylene pellets nominal 2 mm sized with Enviro-Bond 405 polymer and mix thoroughly with a low speed, low shear mixer at ambient temperature. Mixed essential oils together at ambient or sub-ambient temperatures in a separate suitably size vessel compatible with essential oils. After mixing of the polymer and plastic pellets is complete, slowly added essential oil mixture and continue to mix until the essential oils are completely sorbed with the granular polymer/polypropylene pellet mixture and no visible non aqueous phase liquids are visible. The insect repelling essential oil/granular polymer/polypropylene pellet matrix is then packed into a cylindrical permeable polyethylene cylinder with a solid bottom and solid top making the insect repelling device. The insect repelling essential oil/granular polymer/polypropylene pellet matrix can be manufactured either in a batch mode with mixing in a vessel or in a continuous mode with the rate of polypropylene pellets, granular polymer and essential oils added to one or more continuous flow vessels at rates resulting in the same or about the same weight ratios given in FIG. 1. The composition is given in FIG. 1. Various types of mixing devices can be used in either batch or continuous manufacturing operations. Once the insect repelling essential oil/granular polymer/polypropylene pellet matrix is made following the composition and method herein, about 174.49 g of the insect repelling essential oil/polymer/polyethylene pellet matrix is placed in a cylindrical air permeable housing with a volume of about 305.2 cm³ to achieve a packed bulk density of about 0.57 g/cm³. Various sizes of air permeable, air-semi-permeable or air permeable but not liquid water permeable housings can be used to contain the insect repelling essential oil/granular polymer/polypropylene pellet matrix and allow insect repelling volatile organic compounds to be release to the air.

Several insect repelling essential oil/granular polymer/polyethylene pellet matrix filled air permeable housing devices were manufactured according the composition and method herein and placed in a horse barn either in open aisles, a horse stall and in a one-sided open lean-too.

### Example 2

### Insect Repellent Device

DEET (N,N-diethyl-meta-toluamide) is used by an estimated ⅓ of the U.S. population every year, comprising about 27% of adult males, 31% of adult females and 34% of children with about 19% of U.S. households using it on household members and about 4% of households that have cats and/or dogs used DEET on those pets. DEET is used to repel biting flies, biting midges, black flies, chiggers, deer flies, fleas, gnats, horse flies, mosquitoes, no-see-ums, sand flies, small flying insects, stable flies and ticks. DEET products normally are applied directly to skin and/or clothing, are available in numerous formulation types (e.g., aerosol sprays, non-aerosol sprays, creams, lotions, sticks, foams, and towelettes) and concentrations (products range from about 4% active ingredient to 100% active ingredient). The USEPA has concluded that DEET insect repellents will generally not cause unreasonable risks to humans or the environment. DEET has a vapor pressure of 0.0056 mm Hg at 20° C. so it has a volatility that is the range of several essential oil compounds.

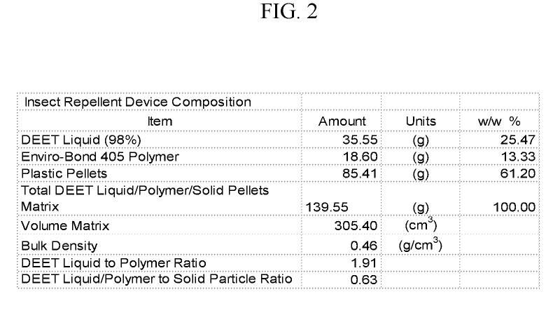

In a suitable sized vessel combine plastic polypropylene pellets nominal 2 mm sized with Enviro-Bond 405 polymer and mix thoroughly with a low speed, low shear mixer at ambient temperature. Mix 95 percent or greater DEET liquid together at ambient or sub-ambient temperatures in a separate suitably size vessel compatible with DEET liquid. After mixing of the granular polymer and plastic pellets is complete slowly add the DEET liquid and continue to mix until the essential oils are completely sorbed with the granular polymer/polypropylene pellet mixture and no visible non aqueous phase liquids are visible. The insect repelling DEET liquid/granular polymer/polypropylene pellet matrix is then packed into a cylindrical permeable polyethylene cylinder with a solid bottom and solid top making the insect repelling device. Batches of the insect repelling DEET liquid/granular polymer/polypropylene pellet matrix can be manufactured either in a batch mode with mixing in a vessel or in a continuous mode with the rate of polypropylene pellets, granular polymer and DEET liquid added to one or more continuous flow vessels at rates resulting in the same or about the same weight ratios given in FIG. 2. The composition is given in FIG. 2. Various types of mixing devices can be used in either batch or continuous manufacturing operations. Once the insect repelling DEET/granular polymer/polypropylene pellet matrix is made following the composition and method of Example 2, about 139.55 g of the insect repelling DEET/granular polymer/polyethylene pellet matrix is placed in a cylindrical air permeable housing with a volume of about 305.4 cm³ to achieve a packed bulk density of about 0.46 g/cm³. Various sized of air permeable, air-semi-permeable or air permeable but not liquid water permeable housings can be used to contain the insect repelling DEET liquid/granular polymer/polypropylene pellet matrix and allow insect repelling DEET volatile organic compound to be release to the air.

Several insect repelling DEET liquid/granular polymer/polyethylene pellet matrix filled air permeable housing devices were manufactured according the composition and method herein and placed in a horse barn either in open aisles, a horse stall and in a one-sided open lean-too.

### Example 3

### Aromatherapy Essential Oil Matrix

In a suitable sized vessel combine plastic polypropylene pellets nominal 2 mm sized with Enviro-Bond 405 polymer and mix thoroughly with a low speed, low shear mixer at ambient temperature. After mixing of the granular polymer and plastic pellets is complete, slowly add Lavandula officinalis essential oil and continue to mix until the essential oil is completely sorbed with the granular polymer/polypropylene pellet mixture and no visible non aqueous phase liquids are visible.

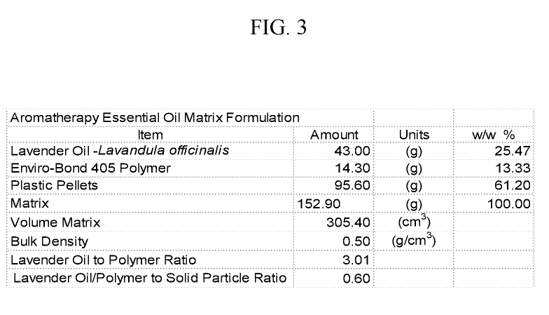

The aromatherapy essential oil/granular polymer/polypropylene pellet matrix is then packed into a cylindrical permeable polyethylene cylinder with a solid bottom and solid top making the aromatherapy volatile organic compound emitting device. Batches of the aromatherapy essential oil/granular polymer/polypropylene pellet matrix can be manufactured either in a batch mode with mixing in a vessel or in a continuous mode with the rate of polypropylene pellets, granular polymer and aromatherapy essential oil added to one or more continuous flow vessels at rates resulting in the same or about the same weight ratios given in FIG. 3. The composition is given in FIG. 3. Various types of mixing devices can be used in either batch or continuous manufacturing operations. Once the aromatherapy essential oil/granular polymer/polypropylene pellet matrix is made following the composition and method of Example 3, about 152.95 g of the aromatherapy essential oil/granular polymer/polyethylene pellet matrix is placed in a cylindrical air permeable housing with a volume of about 305.4 cm³ to achieve a packed bulk density of about 0.50 g/cm³. Various sized of air permeable, air-semi-permeable or air permeable but not liquid water permeable housings can be used to contain the aromatherapy essential oil/granular polymer/polypropylene pellet matrix and allow lavender essential oil volatile organic compound to be released to the air.

Another example of an essential oil volatile organic compound aromatherapy lavender essential oil/polymer/polypropylene pellet matrix emitting device based the formulation of this Example 3 allows the matrix to be packed into a container allowing volatile organic compounds associated with the granular polymer sorbed Lavandula officinalis essential oil to volatilize into the ambient atmosphere from at least one side of a container that allow vapors to escape from the container to the atmosphere. An open container (59.15 mL (2 oz.)) fluid volume container is then filled with 25.98 g of the aromatherapy essential oil/granular polymer/polypropylene pellet matrix from this Example 3 to achieve a bulk density of 0.50. A plastic porous “sifter” insert is placed on the top of the open container and then a screw-top cover is placed and sealed onto the top of the container. When it is desired to release essential oil compounders into the ambient in an environmental around the container, the screw-top cover is removed and evaporation, diffusion and mass transfer of volatile essential oil compounds from the aromatherapy essential oil/granular polymer/polypropylene pellet matrix takes place and volatile organic carbon compounds are released into the atmosphere. When Lavandula officinalis is used in the aromatherapy essential oil/granular polymer/polypropylene pellet matrix of this Example 3, the major aromatic aromatherapy compounds that are volatilized include linalool, linalyl acetate, 1,8-cineole, cis- and trans-linalool oxide, camphor and borneol.

Batches of the aromatherapy essential oil/granular polymer/polypropylene pellet matrix can be manufactured either in a batch mode with mixing in a vessel or in a continuous mode with the rate of polypropylene pellets, granular polymer and essential oils added to one or more continuous flow vessels at rates resulting in the same or about the same weight ratios given in this Example 3. Various types of mixing devices can be used in either batch or continuous manufacturing operations. Various sized of air permeable, air-semi-permeable or air permeable but not liquid water permeable housings can be used to contain the aromatherapy essential oil/granular polymer/polypropylene pellet matrix and allow aromatherapy volatile organic compounds to be release to the air.

### Example 4

### Jasmine Fragrance Matrix

In a suitable sized vessel combine plastic polypropylene pellets nominal 2 mm sized with Enviro-Bond 403 polymer and mix thoroughly with a low speed, low shear mixer at ambient temperature. After mixing of the granular polymer and plastic pellets is complete, slowly add Jasmine Fragrance Oil (purchased from Bulk Apothecary, Aurora, Ohio) and continue to mix until the Jasmine Fragrance Oil is completely sorbed with the granular polymer/polypropylene pellet mixture and no visible non aqueous phase liquids are visible.

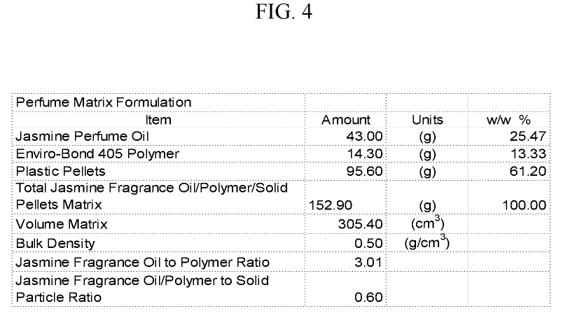

The jasmine fragrance oil/granular polymer/polypropylene pellet matrix is then packed into a cylindrical permeable polyethylene cylinder with a solid bottom and solid top making the perfume volatile organic compound emitting device. Batches of the jasmine Fragrance Oil/granular polymer/polypropylene pellet matrix can be manufactured either in a batch mode with mixing in a vessel or in a continuous mode with the rate of polypropylene pellets, granular polymer and Jasmine Fragrance Oil added to one or more continuous flow vessels at rates resulting in the same or about the same weight ratios given in FIG. 4. The composition is given in FIG. 4. Various types of mixing devices can be used in either batch or continuous manufacturing operations. Once the jasmine fragrance oil/granular polymer/polypropylene pellet matrix is made following the composition and method of this Example 4, about 152.95 g of the jasmine fragrance oil/granular polymer/polyethylene pellet matrix is placed in a cylindrical air permeable housing with a volume of about 305.4 cm³ to achieve a packed bulk density of about 0.50 g/cm³. Various sized of air permeable, air-semi-permeable or air permeable but not liquid water permeable housings can be used to contain the jasmine fragrance oil/granular polymer/polypropylene pellet matrix and allow the perfume volatile organic compound to be released to the air.

Another example of an essential oil volatile organic compound Jasmine Fragrance Oil/polymer/polypropylene pellet matrix emitting device based the formulation of this Example 4 allows the matrix to be packed into a container allowing volatile organic compounds associated with the granular polymer sorbed Jasmine Fragrance Oil to volatilize into the ambient atmosphere from at least one side of a container that allow vapors to escape from the container to the atmosphere. An open container (59.15 mL (2 oz.)) fluid volume container is then filled with 25.98 g of the jasmine fragrance oil/granular polymer/polypropylene pellet matrix from Example 4 to achieve a bulk density of 0.50. A plastic porous “sifter” insert is placed on the top of the open container and then a screw-top cover is placed and sealed onto the top of the container. When it is desired to release essential oil compounders into the ambient in an environmental around the container, the screw-top cover is removed and evaporation, diffusion and mass transfer of volatile essential oil compounds from the jasmine fragrance oil/granular polymer/polypropylene pellet matrix takes place and volatile organic carbon compounds are released into the atmosphere.

Batches of the jasmine fragrance oil/granular polymer/polypropylene pellet matrix can be manufactured either in a batch mode with mixing in a vessel or in a continuous mode with the rate of polypropylene pellets, polymer and jasmine fragrance oil added to one or more continuous flow vessels at rates resulting in the same or about the same weight ratios given in this Example 4. Various types of mixing devices can be used in either batch or continuous manufacturing operations. Various sized of air permeable, air-semi-permeable or air permeable but not liquid water permeable housings can be used to contain the aromatherapy essential oil or fragrance oil/granular polymer/polypropylene pellet matrix and allow aromatherapy volatile organic compounds to be release to the air. Many types of fragrance oils can be used instead of jasmine fragrance oil and that these fragrance oils are readily available and produced. The device in this Example 4 could be used as an air freshener. Aromatherapy essential oils could also be used in this Example 4 with one or more granular polymers and that the device could be used for aromatherapy.

### Example 5

### Active Air Blower Insect Repellent Filter System

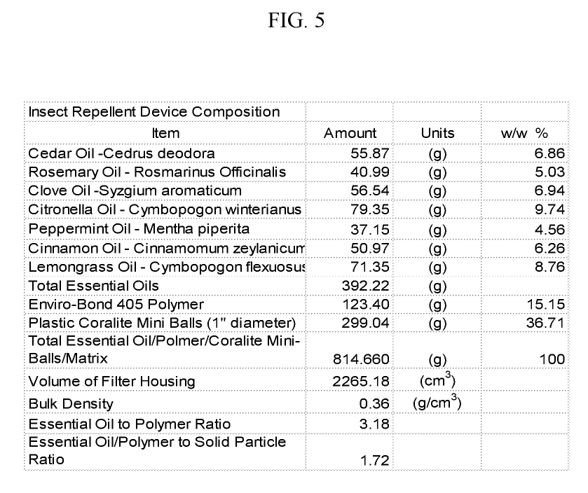

In a suitable sized vessel combine 123.4 g Enviro-Bond 405 polymer and 392.22 g essential oils and mix thoroughly with a low speed, low shear mixer at ambient temperature. Once the granular polymer-essential oil mixture has solidified (about 1 to about 5 minutes) mix in a suitably sized vessel 299.04 g Coralite Mini Bio-Balls (1″ nominal size) with the granular polymer-essential oil mixture at ambient temperature. After mixing of the granular polymer and the Coralite Mini Bio-Balls porous plastic particles is complete and the porosity of the Coralite Mini Bio-Balls is suitably filled with the essential oil/granular polymer/porous plastic particle matrix and the matrix is stable enough so that the matrix does not fall off of the porous Coralite Mini Bio-Balls, the insect repelling essential oil/granular polymer/porous plastic matrix is then packed into a 4 inch (10.16 cm) diameter and 11 inch (27.94 cm) long cylindrical sheet metal filter housing with an empty bed volume of 138.23 in³ (2,265.18 cm³) that has solid gas impermeable sides and open ends covered with aluminum mesh to retain the insect repelling essential oil/granular polymer/porous plastic matrix. Batches of the insect repelling essential oil/granular polymer/porous Coralite Mini Bio-Ball material matrix can be manufactured either in a batch mode with mixing in a vessel or vessels in in a continuous mode with the rate of porous Coralite Mini Bio-Ball material, granular polymer and essential oils added to one or more continuous flow vessels at rates resulting in the same or about the same weight ratios given in FIG. 5. The composition is given in FIG. 5. Various types of mixing devices can be used in either batch or continuous manufacturing operations. Once the insect repelling essential oil/granular polymer/porous plastic particle matrix is made following the composition and method of Example 5, about 814.66 g of the insect repelling essential oil/granular polymer/porous plastic particle matrix is placed in a solid sided-open top and bottom filter housing with a volume of about 138.23 in³ (2,265.18 cm³) to achieve a packed bulk density of about 0.36 g/cm³. Various filter housing materials, various sizes of air filter housings and various composition and porosity mesh tops and bottoms comprising the filter, can be used to contain the insect repelling essential oil/granular polymer/Coralite Mini Bio-Ball material matrix and allow insect repelling volatile organic compounds to be released to the air by means of an air pump or air blower which passes clean air through the filter wherein desorption and volatilization of at least a portion of the volatile organic compounds in the essential oils results in the diffusive and mass transfer flux of granular polymer-sorbed phase volatile organic compounds into a gas or air phase.

In this Example 5, the 4 inch (10.16 cm) diameter filter housing containing the essential oil/granular polymer/porous plastic particle matrix is attached by a flange and bolts to a 115 volt, 2.5 ampere, 0.25 horsepower Air Foxx DB-0250A blower with an air flow capacity of 370 cubic feet per minute. The exhaust end of the filter housing is then attached to a normally closed flapper baffle and then to an air flow directing head. The air blower is connected to a variable control short cycle timer Model 734170 manufactured by Titan Industries, Inc. The timer was adjusted to turn on the blower for 1 minute every 30 minutes during the day and 1 minute every hour during the night time. Various types of timing cycles could be used to control the on/off cycling of the blower motor to optimize insect repellency. A person having ordinary skill in the art would recognize that many sizes, geometries and materials of construction of air filter housings could be used to contain many composition of essential oil/granular polymer mixtures to achieve various type of insect repellency or insecticidal properties. The addition of a fixed oil, such as soybean oil could be used to mix into and dilute the essential oils to decrease effective vapor pressures of the essential oil volatile organic compound as described by Raoult's Law. A person having ordinary skill in the art would recognized that by changing the essential oil mixture or through the addition of a fixed oil in the oil/granular polymer mix, that a filter could be developed that results in the production of an aromatic or fragrant air exiting the air blower or air pump filter housing thereby be transferred into an enclosed, semi-enclosed or open space.

A higher capacity enclosed ring blower, model number KP-680 Koala KOALA B-AIR blower was used with the same filter housing specified in Example 5. The Kaola KP-680 blower has a high capacity than the “squirrel cage” blower used in Example 5. The Kaola KP-680 blower has a 447 cfm capacity and a 6.25 inches static water pressure capacity. This enclosed ring blower was attached to the same filter housing used in example 5 and the exhaust from the filter housing was connected to a 4 inch diameter by 10 foot long cylindrical sheet metal duct that extended vertically to the ceiling of a barn from the filter housing. The exhaust end of the sheet metal duct was connected to a 4″ diameter Tee-connector and each end of the Tee-connector was connected to a horizontal 4 inch diameter by 12 foot long sheet metal duct hung from a wooden ceiling by duct hangers. At the end of each of the two 12 foot horizontal ducts a Tee connector with a damper was connected and enable a controlled flow rate discharged to the ambient air of the semi-enclosed barn. The horizontal ductwork was extended from the Tee connector in a similar fashion in each direction and a set of 90 degree elbows with adjustable dampers were connected to each of the two additional 12 foot sheet metal duct pipe. This system enabled 4 damper controlled air flowrate discharge vents to convey the gas phase volatile organic compounds transferred from the essential oil/granular polymer/porous plastic particle matrix contained in the filter housing when the enclosed ring blower was turned on at a 1 minute interval every 30 minutes during the day and for 1 minute intervals every hour during the night. The flowrates from each of the 4 damper controlled air flowrate vents were adjusted by measuring the air flowrates with a Testo Model 410 vane anemometer. Various air flow rates and static pressure capacity types of air blowers, enclosed ring blowers and air pumps could be used with various sized and geometric configurations of air handling duct work and various types and sizes of air flow control dampers or valves and various geometry, sized and volume capacity filter housings with various essential oil/granular polymer/porous plastic particle matrix to achieve insect repellency, insecticidal, fragrance emitting or aromatherapy emitting volatile organic compounds entering the ambient air.

While we have shown and described several embodiments in accordance with our disclosure, it is to be clearly understood that the same may be susceptible to numerous changes apparent to one skilled in the art. Therefore, we do not wish to be limited to the details shown and described but intend to show all changes and modifications that come within the scope of the appended claims.

1. A device that is manufactured at ambient temperatures used to introduce aromatherapy or fragrance compounds into the air or to repel or inactivate insects or other arthropods by the passive, slow, sustained time-release of insect repelling, insecticidal, aromatherapy or fragrance emitting volatile organic compounds into the air comprising:
a) an aromatherapy, fragrance, perfume, insecticidal or insect repelling essential oil or essential oil extract volatile organic compound-granular polymer matrix consisting of one or more volatile organic compounds sorbed with one or more granular polymers;
b) naturally occurring or man-made porous or non-porous solid particles coated with the volatile organic compound-granular polymer matrix;
c) a gas permeable, gas semi-permeable or a microporous material on at least one side of the device that contains the solid particles coated with the volatile organic compound-polymer-solid particle mixture; and optionally
d) a gas impermeable material on one or more sides of the device that contains the solid particles coated with the volatile organic compound-granular polymer matrix.

2. The device of claim 1, wherein the aromatherapy, fragrance, perfume, insecticidal or insect repelling essential oil or essential oil extract volatile organic compound that provides aromatherapy, fragrance or repels or inactivates insects or other arthropods has a vapor pressure from about 0.0002 mm Hg to about 40.000 mm Hg when measured at a temperature of 20° C. and a pressure of 760 mm Hg.

3. The device of claim 1 wherein the volatile organic compound is an essential oil.

4. The device of claim 3 wherein the aromatherapy, fragrance, perfume, insecticidal or insect repelling essential oil or essential oil extract essential oil is selected from the group consisting of: Abies siberica; Allium sativum, Artemisia spp.; A. vulgaris; A. monosperma; Boswellia caterii; Boswellia serrata; Cananga odorata; Cedar spp; Cedrus atlantica; Cedrus aurantium; Cedrus deodora; Citrus spp; Citrus bergamia; Citrus hystrix; Citrus limonum; Chamaecyparis nootkatensis; Cinnamonium cassia; Cinnamonium comphora; Cinnamomium zeylanicum, Commiphora myrrha; Curcuma longa; Cymbopogon spp.; Cymbopogon citratus; Cymbopogon flexuosus; C. nardus; C. martini; C. citratus; C. winterianius; C. excavates; Corymbia citriodora; Daniellia dived; Eucalyptus spp.; E. camaldulensis; E. globulous; Eugenia aromatica; Eugenia caryophyllus; Gaultheria fragrantissima; Gaultheria procumbens; Juniperus communus; Syzygium aromaticum; Eugenia aromaticum; Lippia spp; L. javanica; L. uckambensis; L. cheraliera; Lantana camara; Lavandula spp.; L. angustifolia; L. latifolia; Leptospermum petersonii; Matricaria recutita; Melaleuca alternifolia; Melissa officinalis; Litsea cubeba; Ocimum spp.; O. americanum; O. suave; O. basilicum; O. kilimandscharikum; O. forskolei; Oreganum vulgare; Hyptis spp.; Hyptis suaveolens; Melaleuca alternifolia; Mentha spp.; M. piperata; M. arvensis; Mentha spicata; Nepeta cataria; Thymus spp.; Th. citriodorus; Th. vulgaris; Th. satureioides; Pelargonium spp.; Pelargonium graveolens; Pimpinella anisum, Pinus sylvestri; Pogostemon spp.; Pogostemon cablin; Pelargonium reniforme; Pseudotsuga menziensii; Azadirachta indica; Rosemarinus officinalis; Salvia officinalis; Salvia sclarea; Syzyguim aromaticum; Tagates spp.; Tagates minuta; Tagates erecta; Thuja orientalis; Vetvivera zizanioides; Zanthoxylum limonella; or Zingiber officinale.

5. (canceled)

6. The device of claim 3 wherein the essential oil or the essential oil extract is selected from the group consisting of: bisabolene; borneol; 3-carene; caryophyllene oxide; camphene; camphor; carvacrol; cis-carveol; cedrol; 1,8-cineole, citral; cis-verbenol, citronellal; citronellol; eugenol; farnesol; geranial; geraniol; 4-isopropylbenzenemethanol; menthol; menthone; methyl salicylate; myrcene; limonene; linalool; p-menthane-3,8-diol; neral; nerol; patchoulol; perillaldehyde; perillyl alcohol; a-pinene; b-pinene; α-terpineol; thymol; vanillin; verbenone; or vetviverol.

7-8. (canceled)

9. The device in claim 1 wherein the granular polymer is a hydrophobic oil sorbing polymer or a hydrophobic oil solidifying polymer.

10. The device in claim 9 wherein the hydrophobic oil sorbing polymers include but are not limited to those selected from the group consisting of: block copolymers, diblock copolymers; triblock copolymers; radial block polymers, multi-block polymers, cross-linked polymers, crosslinked copolymers of hydroxypropyl cellulose acrylate/octadecyl acrylate; cross-linked polyolefin terpolymers; cross-linked octadecylacrylate-co-acrylic acid copolymers; polynorbornene polymers; styrene-ethylene/butylene-sytrene block copolymers; styrene/butadiene block copolymers; polytertiarybutylstyrene; polyacrylo-nitrile butadiene; polyisoprene (rubber), polyethylene; polypropylene; poly isobutylene; polyethylene terephthalate fibers modified by the adherence of crosslinked polymer coatings and materials and polymers sold under the trade name Organolock® N65; Norosorex®; Enviro-Bond™ 403; Enviro-Bond™ 405; Elastol 1; Elastol 2; Nochars A 610; Nochars A 650; Omni-Zorb #2000; Omni-Zorb # AZ1N; Omni-Zorb # BZ; Omni-Zorb # PZ; Petro-Lock; Clear-Tec Rubberizer®, SPI Oil Bond, Waste-set PS #3200, and Waste-set PS #3400.

11. The device of claim 1 wherein the man-made porous or non-porous solid particles are selected from the group of thermoplastic plastic pellets including but not limited to those made with acrylonitrile butadiene styrene (ABS); polyoxymethylene (POM); low density polyethylene (LDPE); high density polyethylene (HDPE); nylon; polyether ether ketone (PEEK); polycarbonate (PC); polyethylene (PE); polypropylene (PP); polystyrene (PS); polyvinyl chloride (PVC).

12. The device of claim 1 wherein the solid particles are made from natural materials selected from the group of natural wood pellets; pea-stone gravel; biomass pellets; coffee husk pellets; hemp pellets; corn cob pellets.

13. The device of claim 1 wherein the solid pellets are either a porous or non-porous with particle sizes varying in length, width and depth dimensions from for example from 1.0 mm to 5.0 mm.

14. The device of claim 1 wherein the gas permeable, gas semi-permeable or microporous materials are selected from the group consisting of gas permeable woven fabrics, non-woven fabrics, injected molded gas permeable plastic mesh materials, gas permeable housings, selected from the group consisting of natural and synthetic fabrics consisting of: wool; cotton; bamboo; hemp; nylon; polyester nylon; polyester; acrylic; rayon; polyolefin; and materials sold under the trade name Gore-Tex®; Tyvek®; Evolution®; or Optima™.

15. The device of claim 1 wherein the optional gas impermeable material is selected from the group consisting of ethylene vinyl alcohol (EVA) polyester laminate; a low-density polyethylene (LDPE) polyester laminate EVA-LDPE polyester laminate; polytetrafluoroethylene (PTFE); high density polyethylene (HDPE); nylon; polyether ether ketone (PEEK); polycarbonate (PC); polyethylene (PE); polypropylene (PP); polystyrene (PS); polyvinyl chloride (PVC); and materials sold under the trade name Evolution®; Compel®; Barrier 450™; and Optima™.

16. The device of claim 1 wherein the ratio of aromatherapy, fragrance, perfume, insecticidal or insect repelling essential oil or essential oil extract volatile organic carbon to granular polymer sorbent is from about 1 to about 15 on a weight basis.

17. The device of claim 1 wherein the ratio of the aromatherapy, fragrance, perfume, insecticidal or insect repelling essential oil or essential oil extract volatile organic carbon plus granular polymer to solid pellet ratio is from about 0.05 to about 3.00 on a weight basis.

18. The device of claim 1 wherein the bulk density of the aromatherapy, fragrance, perfume, insecticidal or insect repelling essential oil or essential oil extract volatile organic carbon/granular polymer/solid particle matrix contained within into the porous housing is from about 0.20 g/cm³ to about 2.0 g/cm³.

19. The device of claim 1 wherein the porous or non-porous solid particles have a packed total porosity from about 0.05 to about 0.60.

20. The device of claim 1 wherein the aromatherapy, fragrance, perfume, insecticidal or insect repelling essential oil or essential oil extract volatile organic carbon/granular polymer/solid particle matrix have an initial packed total porosity from about 0.01 to about 0.55.

21. (canceled)

22. A method to provide aromatherapy, fragrance or to repel or inactivate insects or other arthropods by the passive, slow, sustained time-release of insect repelling or insecticidal, aromatherapy or fragrance emitting volatile organic compounds into the air comprising:
a) an aromatherapy, fragrance, perfume, insecticidal or insect repelling essential oil or essential oil extract volatile organic compound-granular polymer matrix consisting of one or more volatile organic compounds sorbed with one or more solid polymers;
b) naturally occurring or man-made porous or non-porous solid particles coated with the insect repelling, insecticidal, aromatherapy or fragrance emitting essential oil or essential oil extract volatile organic compound-granular polymer matrix;
c) a gas permeable, gas semi-permeable or a microporous material on at least one side of the structure that contains the solid particles coated with the insect repelling, insecticidal, aromatherapy or fragrance emitting essential oil or essential oil extract volatile organic compound-granular polymer-solid particle mixture; and optionally
d) a gas impermeable material on one or more sides of the material that contains the solid particles coated with the insect repelling, insecticidal, aromatherapy or fragrance emitting essential oil or essential oil extract volatile organic compound-granular polymer matrix.

23-96. (canceled)

97. A device that is manufactured at ambient temperatures used to introduce aromatherapy or fragrance compounds into the air or to repel or inactivate insects or other arthropods by the active pumping of air through an air filter for the controlled, sustained time-release of insect repelling, insecticidal, aromatherapy or fragrance emitting volatile organic compounds into the air comprising:
a) an aromatherapy, fragrance, perfume, insecticidal or insect repelling essential oil or essential oil extract volatile organic compound-granular polymer matrix consisting of one or more volatile organic compounds sorbed with one or more solid polymers;
b) naturally occurring or man-made porous or non-porous solid particles coated with the volatile organic compound-granular polymer matrix;
c) a gas permeable material on at least two sides of a filter housing that contains the solid particles coated with the volatile organic compound-polymer-solid particle mixture; and optionally
d) a gas impermeable material on one or more sides of the filter housing that contains the solid particles coated with the volatile organic compound-granular polymer matrix.

98-138. (canceled)

139. A method to provide aromatherapy, fragrance or to repel or inactivate insects or other arthropods by the active pumping of air through an air filter for the controlled, sustained time-release of insect repelling, insecticidal, aromatherapy or fragrance emitting volatile organic compounds into the air comprising:
a) an aromatherapy, fragrance, perfume, insecticidal or insect repelling essential oil or essential oil extract volatile organic compound-granular polymer matrix consisting of one or more volatile organic compounds sorbed with one or more solid polymers;
b) naturally occurring or man-made porous or non-porous solid particles coated with the volatile organic compound-granular polymer matrix;
c) a gas permeable material on at least two sides of a filter housing that contains the solid particles coated with the volatile organic compound-polymer-solid particle mixture; and optionally
d) a gas impermeable material on one or more sides of the filter housing that contains the solid particles coated with the volatile organic compound-granular polymer matrix.

140-287. (canceled)
